(19) United States
(12) Patent Application Publication
(10) Pub. No.: US 2020/0372972 A1
(43) Pub. Date: Nov. 26, 2020

(54) SAMPLE DATA ANALYSIS METHOD BASED ON GENOMIC MODULE NETWORK

(71) Applicant: Industry-University Cooperation Foundation Hanyang University, Seoul (KR)

(72) Inventors: Jin Hyuk KIM, Seongnam-si (KR); Hye Young KIM, Seongnam-si (KR)

(73) Assignee: Industry-University Cooperation Foundation Hanyang University, Seoul (KR)

(21) Appl. No.: 16/635,433

(22) PCT Filed: Oct. 25, 2018
(86) PCT No.: PCT/KR2018/012678

§ 371 Date: Jan. 30, 2020

(30) Foreign Application Priority Data
Nov. 13, 2017 (KR) 10-2017-0150582
Feb. 7, 2018 (KR) 10-2018-0015031
Jun. 19, 2018 (KR) 10-2018-0070492

(51) Int. Cl. G16B 40/00; G16B 25/10; G16B 5/20

(52) U.S. Cl. CPC G16B 40/00; G16B 5/20; G16B 25/10

(57) ABSTRACT

Provided is a method of analyzing sample data based on a genomic module network by means of a computer apparatus. The method includes filtering first gene expression data for a normal or tumor tissue, which is the same tissue as a specific tissue, and second gene expression data for a target tissue to be analyzed, which is the same tissue as the specific tissue, on the basis of a specific module among a plurality of genomic modules; and classifying genes into a plurality of new genomic modules on the basis of an entropy determined using the filtered first gene expression data and determining, for genes belonging to at least one of the plurality of new genomic modules, a first degree of variation of the target tissue relative to the normal or tumor tissue in the at least one genomic module using the filtered first gene expression data and the filtered second gene expression data.

### CROSS REFERENCE TO RELATED APPLICATIONS

This application is a National Stage of International Application no. PCT/KR2018/012678, filed Oct. 25, 2018, claiming priority based on Korean Patent Application No. 10-2017-0150582 filed Nov. 13, 2017, Korean Patent Application No. 10-2018-0015031 filed Feb. 7, 2018 and Korean Patent Application No. 10-2018-0070492 filed Jun. 19, 2018.

### TECHNICAL FIELD

The following description relates to a sample data analyzing technique based on the genomic module network of which applies to the diagnosis and/or prognosis of cancer.

### BACKGROUND ART

The etiology of cancer, such as malignant tumors, is conventionally presumed to be in genomes in general. Thus, most cancer studies focus on the genome. With the advancement of molecular biology, molecular-targeted therapies have developed for selectively killing cancer cells and reducing the side effects of conventional anticancer chemotherapy. Studies on cancer treatment are yet incomplete due to a lack of understanding of functions and mechanisms of the genome. The conventional genome studies dependent on biochemical techniques have limits on expanding the understanding of the genome, beyond the chemical reactions and structures.

### DETAILED DESCRIPTION OF THE INVENTION

### Technical Problem

The following description provides a genomic module network generating a technique for sample data analysis. The following description also provides a new analyzing technique for sample data based on a genomic module network constructed with gene expression data from a specific tissue. The following description also accommodates indicators that represent a state of a specific sample based on the criteria from the constructed genomic module network.

### Technical Solution

In one general aspect, there is a genome analyzing method based on modularization via a computer apparatus includes (i) receiving gene expression data of a specific tissue, (ii) calculating entropy levels of a plurality of gene sets among the genome from the gene expression data, (iii) identifying a plurality of genomic modules with a plurality of the gene sets based on the entropy, and (iv) generating a genomic module network with determining on edges which connect genomic modules each other based on relative entropy of each genomic modules.

In another general aspect, there is a sample data analyzing method based on genomic module network via computer apparatus includes the following steps: (1) identifying a plurality of genomic modules with a plurality of gene sets included in a genome of a specific tissue by calculating entropy from the gene expression data of the specific tissue, (2) filtering the first gene expression data of normal and/or tumor tissue, i.e., the same kind of tissue as a specific tissue, and the second gene expression data of a target tissue, i.e., a subject of analysis, the same kind of tissue as a specific tissue, based on a specific genomic module among a plurality of the genomic modules, (3) identifying a plurality of new genomic modules with a plurality of gene sets included in a genome of a specific tissue by calculating entropy from the filtered first gene expression data of the specific tissue, and (4) determining the degree of transformation of the target tissue against the normal and/or tumor tissue in at least one of a plurality of the genomic modules, using the filtered first gene expression data and the filtered second gene expression data based on at least one of a plurality of the new genomic modules.

### Advantageous Effects of the Invention

The following description of the technique could analyze a genome based on the network structure and information flows so that it can provide appropriate treatment policy for individual patients of different types of disease. The following description of the technique also could provide a solution to the sample analysis based on genomic module network constructed with a gene expression dataset. Thus, it accommodates biological analysis and/or diagnosis for any sample.

### DESCRIPTION OF THE DRAWINGS

The patent or application file contains at least one drawing executed in color. copies of this patent or patent application publication with color drawings will be provided by the Office upon request and payment of the necessary fee.

FIG. 1 illustrates an example of a genomic module network.

FIG. 2 illustrates an example of basis state of a genome composed of two genes.

FIG. 3 illustrates an example of a density matrix in a genome space.

FIG. 4 illustrates an example of genomic modules located in a matrix of basis states for all genes in a genome.

FIG. 5 illustrates an example of a density matrix and a probability of a gene in a genome space and a sample space.

FIG. 6 illustrates an example of genetic networks of a kernel module isolated from each of eight different types of tissue.

FIGS. 7A-7H illustrate an example of a genetic network of the kernel module of normal breast tissue (BRNO) and the mapping of the kernel module into other tissue types.

FIG. 8 illustrates an example of the mapping of genomic modules in a cell cycle and DNA repair (CCDR) domain of BRNO into other tissue types.

FIG. 9 illustrates an example of a density matrix of a genomic module in a sample space.

FIGS. 10A and 10B illustrate an example of the change in genetic network of a genomic module by a perturbation of exclusion of gene i.

FIG. 11 illustrates an example of intermodular networks of eight different tissues based on TCGA dataset.

FIG. 12 illustrates an example of intermodular networks of BRNO at various cutoffs.

FIGS. 13A-13D illustrate an example of an intermodular network of BRNO mapped to other types of tissue.

FIG. 14 illustrates an example of a flowchart for a sample data analysis method based on a genomic module network.

FIG. 15 illustrates an example of a flowchart for a genomic module network construction process.

FIG. 16 illustrates an example process of generating analysis indicators for sample data based on a genomic module network.

FIG. 17 illustrates an example of sample probabilities (SPs) of a plurality of tumor tissue samples based on a plurality of genomic modules of normal tissue.

FIGS. 18A and 18B illustrate an example of the survival analysis in tumor sample groups classified based on SPs.

FIG. 19 illustrates an example of modular sample probabilities (MSPs) of a plurality of tissue samples based on each genomic module of normal tissue.

FIG. 20 illustrates an example of tumor tissue samples classification based on MSPs.

FIG. 21 illustrates an example of the average MSP of each tumor sample group depicted on an intermodular network of normal tissue.

FIG. 22 illustrates an example of MSPs of typical tumor samples belonging to different tumor sample groups.

FIG. 23 illustrates an example of a density matrix and probability locus of a genomic module in a gene space.

FIG. 24 illustrates an example of box plot of log odds ratios (LORs) versus genes in each tumor sample using a density matrix of the gene group of normal tissue.

FIG. 25 illustrates an example of dot plot of LORs versus tumor samples calculated for each gene using a density matrix of a genomic module of normal tissue.

FIGS. 26A and 26B illustrate an example of LORs and log expression ratios (LERs) of genes in each sample of normal and tumor tissues based on a specific genomic module of normal tissue.

FIG. 27 illustrates an example of flowchart for a sample data analysis method based on based on genomic module networks constructed with a filtered gene expression dataset.

FIG. 28 illustrates an example process of generating analysis indicators for sample data based on genomic module networks constructed with a filtered gene expression dataset.

FIG. 29 illustrates an example of process for generating a filtered gene expression dataset.

FIGS. 30A and 30B illustrate an example of a genomic module network based on a filtered BRCA gene expression dataset.

FIGS. 31A and 31B illustrate an example of samples classified based on an MSP at a specific module of the genomic module network in FIGS. 30A and 30B.

FIG. 32 illustrates another example of samples classified based on an MSP at a specific module of the genomic module network in FIGS. 30A and 30B.

FIGS. 33A and 33B illustrate an example of survival analysis for specific sample groups.

FIGS. 34A and 34B illustrate an example of sample classification based on MSPs at another module of the genomic module network in FIGS. 30A and 30B.

FIG. 35 illustrates another example of sample classification based on MSPs at another module of the genomic module network in FIGS. 30A and 30B.

FIGS. 36A and 36B illustrate of survival analysis for sample groups classified in FIG. 35.

FIGS. 37A and 37B illustrate an example of genomic modules for classifying breast cancer samples of lymphocyte infiltration among the intermodular network in FIGS. 30A and 30B.

FIGS. 38A and 38B illustrate an example of genomic modules for classifying breast cancer samples of lymphocyte infiltration among the BRCA intermodular network.

FIG. 39 illustrates an example of survival analysis on sample groups classified using a breast cancer latent genomic module in FIGS. 30A and 30B and a genomic module of different dataset.

FIG. 40 illustrates an example of an intermodular network based on a filtered BRNO gene expression dataset.

FIGS. 41A and 41B illustrate an example of a genomic module of stem cell-like cells in the BRCA genomic module network.

FIG. 42 illustrates an example of estimated modules of a stem cell-like cell in BNRF genomic module network.

FIG. 43 illustrates an example of samples classification based on MSPs at the genomic module network in FIGS. 41A, 41B and FIG. 42.

FIGS. 44A-44C illustrate an example of a sample data analysis process based on a genomic module network.

FIGS. 45A-45C illustrate an example of a sample data analysis apparatus based on genomic module network.

### MODE OF INVENTION

Specific embodiments will be shown in the accompanying drawings and be described in detail below because the following description may be variously modified and have several example embodiments. It should be understood, however, that there is no intent to limit the following description to the particular forms disclosed, but on the contrary, the invention is to cover all modifications, equivalents, and alternatives falling within the spirit and scope of the following description.

Unless defined otherwise, technical and scientific terms used herein have the same meaning as commonly understood by one of ordinary skill in the art. For (the purposes of) the present invention, the following terms are defined below.

(1) The term “sample” as used herein can refer to an individual living organism, an individual patient of a specific disease, or a population of cells procured at a timepoint.

(2) The term “sample data” as used herein can refer to a gene expression data of a sample.

(3) The term “gene expression” as used herein can refer to the transcription of a gene into RNA products.

(4) The term “gene expression data” as used herein can refer to a set of data that contains the gene expression levels of a plurality of genes of a sample measured via high-throughput technology (e.g., microarrays).

(5) The term “gene expression dataset” or “dataset” as used herein can refer to a set of data that contains the gene expression data of a plurality of samples of the same tissue type.

(6) The term “genomic module” or “module” as used herein can refer to a group of genes engaged in one of simultaneous duties of the genome of a high multicellular eukaryotic organism. One genomic module consists of a plurality of genes.

(7) The term “modularization” as used herein can refer to the entire process of finding a plurality of genomic modules using a gene expression dataset.

(8) The term “intermodular network” as used herein can refer to a network in which a plurality of genomic modules are connected by edges.

(9) The term “genetic network” as used herein can refer to a network within a module where genes connected by edges.

(10) The term “edge” as used herein can refer to a channel that can exchange or transfer information between genomic modules in an intermodular network or between genes in a genetic network.

(11) The term “genomic module network” as used herein can refer to a network in general including an intermodular network and genetic networks.

(12) The term “domain” or “genomic module domain” as used herein can refer to a specific region of an intermodular network, composed of a plurality of genomic modules having the common biological functions.

(13) The term “mapping” as used herein can refer to an operation that transfers a list of a plurality of genes in a genomic module or a genomic module network of a given dataset to another dataset and executes the corresponding analysis (e.g. calculation of the entropy, and reconstruction of a genomic module network).

(14) The term “genome space” as used herein can refer to a Hilbert space defined with the basis state vectors of a genome as the coordinate axes.

(15) The term “sample space” as used herein can refer to an m-dimensional space defined with m samples in a given dataset of analysis as the coordinate axes.

(16) The term “gene space” as used herein can refer to an n-dimensional space defined with n genes in a given dataset of analysis as the coordinate axes.

(17) The term “modular sample probability” or “MSP” as used herein can refer to the probability of each sample to a plurality of genes in a specific genomic module.

(18) The term “domain sample probability” or “DSP” as used herein can refer to the probability of each sample to a plurality of genes in a plurality of genomic modules in a specific domain.

(19) The term “sample probability” or “SP” as used herein can refer to the probability of each sample to a plurality of genes in a whole genomic module network.

(20) The term “degree of transformation of the genome system” or “degree of transformation” as used herein can refer to a quantitative value indicating the modification or disintegration of a specific genomic module, a specific domain, or a whole genomic modular network of an individual sample (e.g. MSP, DSP, and SP).

(21) The term “log odds ratio” or “LOR” as used herein can refer to the logarithm of the ratio of the probability of a gene in a genomic module in the absence of a specific gene to the probability of the same gene in the presence of the given gene.

(22) The term “LOR_{MSP}” as used herein can refer to the negative logarithm of the ratio of the MSP of a sample in the absence of a specific gene to the MSP of the same sample in the presence of the given gene.

(23) The term “LOR_{DSP}” as used herein can refer to the negative logarithm of the ratio of the DSP of a sample in the absence of a specific gene to the DSP of the same sample in the presence of the given gene.

(24) The term “LOR_{SP}” as used herein can refer to the negative logarithm of the ratio of the SP of a sample in the absence of a specific gene to the SP of the same sample in the presence of the given gene.

(25) The term “principal eigenvector” as used herein can refer to the eigenvector having the largest eigenvalue as a result of the singular value decomposition (SVD).

(26) The term “kernel module” as used herein can refer to a genomic module having an entropy level lower than other modules in both the original tissue and any other type of tissue when the mapping was applied.

The following description is for revealing the relationship between a genomic module network and a phenotype. We search for information flows on the transcription activity of the genome and confirm them in terms of its phenotype for the purpose.

Biochemical techniques are prevalent in most of the conventional studies on the disease (e.g. malignant tumors). The following example described herein is distinct from the traditional biochemical techniques in terms of perspective on living organisms. This new technique analyzes living organisms as one system.

Living organisms have evolved by forming their complex structures in both vertical and horizontal ways: from anucleate cells to nucleated cells and from unicellular organisms to multicellular organisms. Throughout the vertical and horizontal evolution, living organisms develop multilayered structures and form a complex network system between components. In general, a system is an aggregate of components interconnected by the integrated way; each component or a set of components affects the properties of the system. Ackhoff (1972) and Checkland (1981) suggested that a system expresses its properties rather than the property of an individual component or a part. As the principle above, the biological system expresses the properties of their system instead of proteins and genes. The expression of the properties of a biological system may result in the phenotype. Genes and proteins can affect the phenotypes of living organisms; however, they are not the key components of the biological system itself.

The biological systems respond to internal and external environmental challenges by expressing appropriate phenotypes. This response scenario can be encoded in DNA chains only; thus, the information transmitting via genes determines the phenotypes of the whole biological system.

The direct cause of death for malignant tumors does not come from the changes in expression levels of specific genes and proteins; it comes from phenotypes of malignant tumors and/or cancer cells. Since the phenotype of malignant tumor emerges from the properties of the cancerous biological system itself, it is difficult to regulate or interrupt the expression of the phenotype. The following description is intended to extract genetic information associated with a specific phenotype of a malignant tumor and identify relationships between the genetic information and phenotypes of a biological system. The following description uses a genomic module network obtained by modularizing genes associated with a phenotype. The genomic module network is a model for modularizing the genes associated with the phenotype according to a certain criteria and defining an interrelationship between the modules.

A computer apparatus may perform the genomic module network construction and the analysis based on the genomic module network. The computer apparatus can refer to an apparatus capable of computing or processing input data in a uniform manner. For example, the computer apparatus may be any apparatus such as a personal computer (PC), a smartphone, a server, and the like.

The following description relates to the construction of a genomic module network in a living organism, or associated with a specific disease, using gene expression data. The basic concepts and techniques for constructing the genomic module network will be described below. The genomic module network is one system.

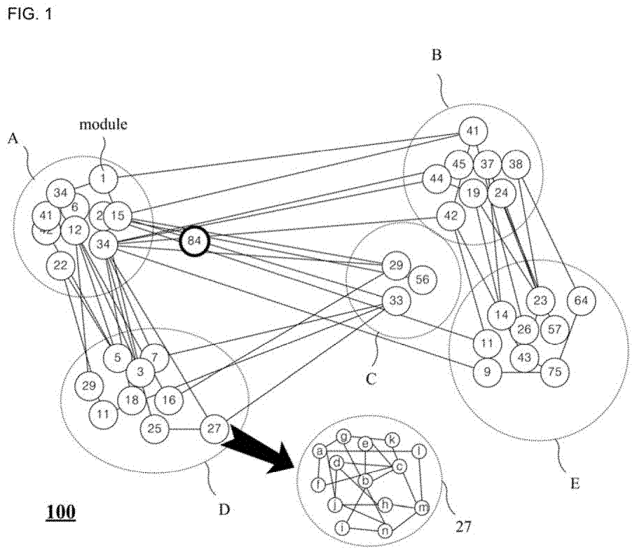

FIG. 1 illustrates an example of a genomic module network 100. First, the structure of the genomic module network 100 will be described before a process of constructing the genomic module network 100 is described. The genomic module network is composed of a plurality of genomic modules and edges connecting certain modules.

Each module of the genomic module network 100 includes certain genes. In FIG. 1, a solid line circle represents each genomic module. The number on the circle denotes the genomic module indicator. Genes belonging to a genomic module have a strong relationship with a specific phenotype of the biological system. The plurality of genomic modules may be separated into groups that perform certain functions concerning the phenotype. In FIG. 1, a dotted line circle denotes a genomic module domain. The domains A, B, C, D, and E are shown in FIG. 1. The module 84 serves as an intermediate that connects domain A and domain C. Each domain is related to a specific biological function of the genome in tissues composed of a plurality of heterogeneous cells, such as cell cycle regulation and DNA repairs, epithelia formation, extracellular matrix formation, immune response, and angiogenesis.

FIG. 1 shows that a solid line (edge) connects any pair of modules. When an edge connects two modules, the modules have a certain relationship with each other. When the two modules are connected by an edge, it indicates that one module involves in the function of another module. When the modules that belong to different domains are connected by an edge, it indicates that one domain involves in the function of another domain. A specific phenotype can be expressed by a module, and several various modules can be directly or indirectly involved in the expression of a specific phenotype.

FIG. 1. shows an enlargement of module 27. Each module is composed of a plurality of genes. Alphabet letter identifies the genes in module 27. An edge connects a pair of genes in the module. The genes in a module form a network similar to the formation of the intermodular network. This is called a genetic network.

Hereinafter, a process of constructing the genomic module network includes a process of modularizing a genome, a process of constructing a network between genomic modules (i.e. intermodular network), and a process of constructing a genetic network in each module. Each process will be described below.

[symbol]

State of Genome

First, a concept for defining the state of a genome will be described. The state of a genome is described at the level of a quantum system. The quantum system could be represented as a density matrix.

A gene can exist in one of two basis states, i.e., an active state and an inactive state. The active state indicates that a corresponding gene is active during a transcription process. At a specific time point, one gene exists in one of the active state or the inactive state. Both states are mutually exclusive, and mathematically orthogonal to each other in a vector space. The active state may be represented by “1” or “on,” and the inactive state may be represented by “0” or “off.” The active state and the inactive state may be represented by basis state vectors |1 [symbol] and |0 [symbol] , respectively. A real state vector |g [symbol] of the gene is a linear combination of the two basis states as described in Equation 1 below.

[symbol]

|g [symbol] =a₀|0 [symbol] +a₁|1 [symbol] . [Equation 1]

In Equation 1, a₀ is a coefficient for the inactive state and a₁ is a coefficient for the active state. The quantity of mRNA generated by the gene depends on a₁. An active state vector |g* [symbol] of the gene is a₁|1 [symbol] , and may be described in a generalized equation as Equation 2 below.

$$\vert {g}^{*} 〉 = {\sum }_{j = 0}^{1} {ja}_{j} \vert j 〉 . \qquad [ Equation 2 ]$$

The basis state vectors |1 [symbol] and |0> [symbol] orthonormal. The active state and the inactive state may be normalized with respect to the active state. For example, two genetic states |g [symbol] [symbol] g| may be normalized with respect to one of both states. A coefficient for the state may be normalized as a₁²(a₀²+a₁²). A coefficient for |1 [symbol] [symbol] 1| indicates a possibility of an active state for a gene.

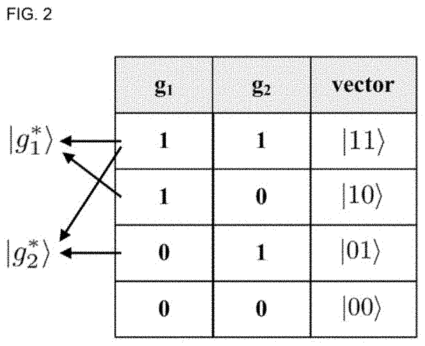

FIG. 2 illustrates an example of basis states of a genome composed of two genes. FIG. 2 shows basis states of two genes g₁ and g₂. The two genes may exist in one of four types of basis states. FIG. 2 shows active state vectors |g*₁ [symbol] and |g*₂ [symbol] for the genes g₁ and g₂, respectively.

When a genome consists of n genes, the whole genes of the genome may have 2ⁿ basis states. If n=2, each gene has two orthonormal basis state vectors. The basis state vectors of the genome may be represented by |j₁j₂ [symbol] , where, jᵢÅ{0,1} for i=1,2. A genome consisting of two genes have four orthonormal basis state vectors as |00 [symbol] , |01 [symbol] , |10 [symbol] , and |11≈. A new vector space may be spanned based on the number of genes. The space defined by basis state vectors of a genome is a Hilbert space. The space defined by basis state vectors of a genome is referred to as a genome space.

The real states |gᵢ [symbol] of a gene in a two-gene genome may be formulated as Equation 3 below. The two genes include a first gene and a second gene.

$$\begin{matrix}\vert {g}_{i} 〉 = ( {a}_{i {0}_{1}} \vert {0}_{1} 〉 + {a}_{i {1}_{1}} \vert {1}_{1} 〉 ) ⊗ ( {a}_{i {0}_{2}} \vert {0}_{2} 〉 + {a}_{i {1}_{2}} \vert {1}_{2} 〉 ) \\ = {c}_{i 00} \vert 00 〉 + {c}_{i 01} \vert 01 〉 + {c}_{i 10} \vert 10 〉 + {c}_{i 11} \vert 11 〉 \\ = {\sum }_{{j}_{1} , {j}_{2} = 0}^{1} {c}_{{ij}_{1} {j}_{2}} \vert {j}_{1} {j}_{2} 〉 , where i = 1 , 2.\end{matrix} . \qquad [ Equation 3 ]$$

The real state of a gene may be determined by the basis states of all the genes in the genome. In Equation 3, aᵢ₀_{₁} is a coefficient for the inactive state of the first gene, and aᵢ₁_{₁} is a coefficient for the active state of the first gene. Also, aᵢ₀_{₂} is a coefficient for the inactive state of the second gene, and aᵢ₁_{₂} is a coefficient for the active state of the second gene. The two genes may have all four types of basis states. cᵢ₀₀ is a coefficient when both of the two genes are inactive. cᵢ₀₁ is a coefficient when the first gene is inactive and the second gene is active. cᵢ₁₀ is a coefficient when the first gene is active and the second gene is inactive. cᵢ₁₁ is a coefficient when both of the two genes are active.

The active state of gene i is |g*ᵢ [symbol] =Σⱼ_{₁}_{,j}_{₂}jᵢcᵢⱼ_{₁}_{,j}_{₂}|j₁j₂ [symbol] . Gene i takes a level of transcription A dyad of the state of a gene may provide the probabilities of dwelling in the basis state, as described in Equation 4 below.

$$\vert {g}_{i} 〉 〈 {g}_{i} \vert = {\sum }_{{j}_{1} , {j}_{2} = 0}^{1} {c}_{{ij}_{1} {j}_{2}}^{2} \vert {j}_{1} {j}_{2} 〉 〈 {j}_{2} {j}_{1} \vert . \qquad [ Equation 4 ]$$

In Equation 4, cᵢⱼ_{₁}ⱼ_{₂}² indicates the probability of gene i dwelling in the basis state |j₁j₂ [symbol] . cᵢⱼ_{₁}ⱼ_{₂}² may be determined by diag(|gᵢ [symbol] [symbol] gᵢ|) for selecting a diagonal element of a matrix. When gene i stays in the active state, the probabilities may be determined by diag(|g*ᵢ [symbol] [symbol] g*ᵢ|). The probability of the whole genome dwelling in |j₁j₂ [symbol] equals the sum of the probabilities of each gene in the genome dwelling in the state.

The probability distribution of the states of the genes in the genome represents a relationship between the genes. When the genes have a uniform distribution, the genes may have random activity without correlation. However, as the correlation between the genes increases, the unevenness of the state distribution of the genes increases. Accordingly, the unevenness of the probability distribution of the genetic states may refer to information indicating correlation of a corresponding gene in the genome.

[symbol] When a genome consists of n genes, there are 2ⁿ basis states for the whole genome. The α th basis state of the genome is simplified as |ψ_{α} [symbol] . |ψ_{α} [symbol] represents |j_{1α} . . . j_{iα} . . . j_{nα} [symbol] ∈{|0 . . . 0 [symbol] , . . . , |1 . . . 1 [symbol] } where j_{iα}(∈{0,1}) is the α th basis state of gene i and all the |ψ_{α} [symbol] 's are mutually orthonormal. Therefore, the active state of gene i may be described as Equation 5 below.

$$\vert {g}_{i}^{*} 〉 = {\sum }_{α = 1}^{{2}^{n}} {j}_{i α} {c}_{i α} \vert {ψ}_{α} 〉 . \qquad [ Equation 5 ]$$

[symbol]

The degree of mRNA generation of gene i depends on the coefficient Σ_{α}j_{iα}c_{iα.} Therefore, the whole genome controls a gene to control generation of mRNA.

[symbol]

A dyad |g*ᵢ* [symbol] [symbol] g*ᵢ| normalized to have trace 1 can be called a density matrix ρᵢ of gene i. Since ρᵢ² is equal to ρᵢ, the density matrix indicates a pure state of the genome. In a quantum system, the pure state indicates that the states are accurately known. Considering the stochastic nature of the genomic system, it is useful to adopt a density matrix to describe a mixed state of a genome as an ensemble of pure states of genes. Therefore, a mixed state density matrix ρ of the genome is given by an ensemble of ρᵢ. That is, ρ is Σᵢ₌₁ⁿωᵢρᵢ, where ωᵢ is the probability of ρᵢ. When ω has the same value, i.e., 1/n, ρ may be formulated as Σᵢ|g*ᵢ [symbol] [symbol] g*ᵢ|. Therefore, ρ may be described by Equation 6 below.

$$ρ = \frac{{Σ}_{i , α} {j}_{i α}^{2} {c}_{i α}^{2} \vert {ψ}_{α} 〉 〈 {ψ}_{α} \vert}{{Σ}_{i , α} {j}_{i α}^{2} {c}_{i α}^{2}} . \qquad [ Equation 6 ]$$

Since a genome space is a Hilbert space, the probability of any unit vector |u [symbol] for the density matrix ρ may be defined according to the Gleason's theorem, as described in Equation 7 below.

Tr(ρ|u [symbol] [symbol] u|)= [symbol] u|ρ|u [symbol] [Equation 7]

The dwelling probability of the genome in an a th basis state is given by [symbol] ψ_{α}|ρψ_{α} [symbol] . This probability may be calculated as Σᵢj_{iα}²c_{iα}²/Σ_{i,α}j_{iα}²c_{iα}². The dwelling probabilities of the genome in a specific basis state are diagonally arranged in the density matrix of the genome. As the density matrix of a genome consisting of n genes is a 2ⁿ×2ⁿ square matrix. The density matrix has 2ⁿ eigenvectors and 2ⁿ eigenvalues. The eigenvectors indicate eigenstates, and the eigenvalues indicate dwelling probabilities of specific states.

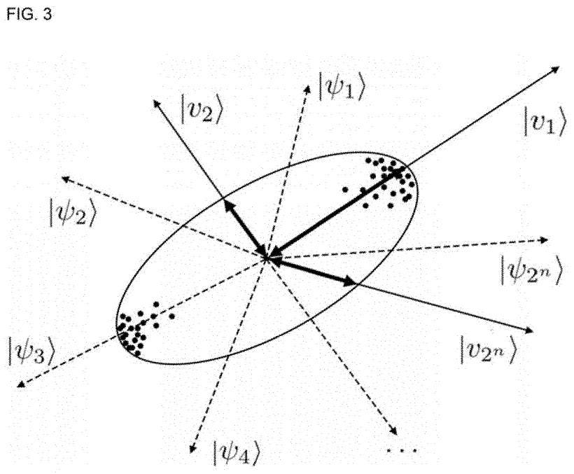

The unevenness of probabilities dwelling in corresponding eigenstates should be considered genetic information generated by the genomic system. FIG. 3 illustrates an example of a density matrix in a genome space. The density matrix is an ellipsoid in a two-dimensional genome space. 2ⁿ axes |ψ [symbol] denoted by dotted arrows represent the basis state vector of a genome. 2ⁿ axes |ν [symbol] denoted by solid arrows represent eigenvectors. The length of thick arrows denotes the probabilities of the eigenvectors |ν [symbol] . Black dots indicate genes.

The eigenvector of the mixed state density matrix ρ specify the properties of emergent traits, and the eigenvalues of the eigenvectors determine the probability of their emergences.

The unevenness may be given by von Neumann entropy S(ρ). Here, entropy means an average of information contents in nats (i.e., a unit of information). A high value of entropy means that the genome can activate genes in no specific interaction pattern or too many interaction patterns engaged in diverse emergent traits. As entropy increases in the genome space, the ellipsoid of the density matrix becomes circular in the genome space and so loses its directionality. On the other hand, a low value of entropy means the genome must be concentrated on a few specific targets. The genetic information generated in the genome space can be transmitted to a protein network in a real space. The mRNA can play a role as parallel channels at the interface between the genome space and the protein space.

[symbol]

Genome Modularization

High multicellular eukaryotic organisms can simultaneously activate different protein networks even in a single cell. It is assumed that genes involved in a specific interaction belong to one group. A group of genes engage in correlation with each other in order to generate a protein contributing a phenotype related to a specific interaction. Therefore, such groups of genes may be defined as genomic modules. A genomic module consists of genes is involved in generating a protein for a specific phenotype. When the genes of the whole genome are analyzed, the genome may be divided into a plurality of modules. The genes belonging to the module may be directly involved in generating a protein for a specific phenotype. Furthermore, the genes belonging to the module may be indirectly involved in a process of generating a specific protein.

The researchers divide the whole genome into as many independent modules as possible, analyze correlations between the independent modules, and find out edges (links) between the modules. The edge describes a relation between two modules. A network in which the whole genome is defined by the modules and the edges may be called as a genomic module network. A genome can be analyzed by the genomic module network.

A plurality of modules may be cooperatively involved in the expression of a specific phenotype. A plurality of modules may perform certain communication through edges between the modules.

In principle, it is possible to isolate genomic modules through proper sorting of basis states and gene indices. The dwelling probability of the genome staying in each basis state should be almost close to zero for the most part but fluctuate in spectrums of genomic modules.

In the case of single cell, a gene can play only one role because the gene cannot dwell simultaneously in multiple different states. In other words, mRNA maintains a single level in physically continuous spaces, it is reasonable to include a gene in only one genomic module.

On the other hand, in the case of a multicellular organism, one gene is expressed in physically separated spaces. In a eukaryotic organism, a gene may perform multitasking by space division corresponding to multitasking of a central processing unit of a computer through time division.

[symbol]

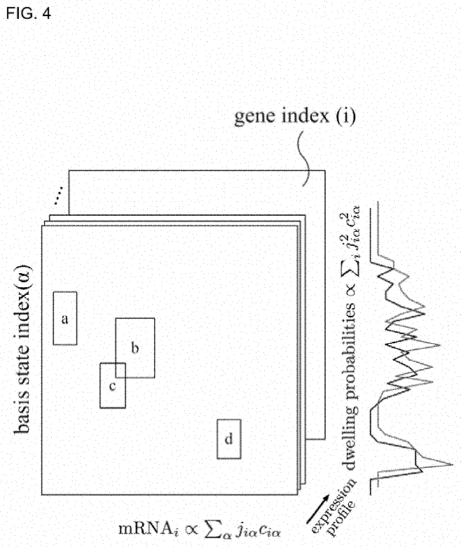

FIG. 4 illustrates an example of genomic modules located in a matrix of basis states for all genes in a genome. The vertical axis indicates basis state indices, and the horizontal axis indicates gene indices. Module c and module b partially overlap each other.

Modules a, b, and d or modules a, c, and d may be activated in one cell (a single genome space). However, modules b and c should be activated in different cells (multiple genome spaces).

Modules a and b have partially overlapping basis states, but the eigenvectors of the two modules have different directionality. Accordingly, the two modules, i.e., modules a and b are involved in different protein networks and phenotypes. Mutual information I(a:b) of both modules, i.e., modules a and b may be represented by S(ρₐ)+S(ρ_{b})−S(ρ_{ab}). Mutual information indicates mutual dependency between both modules. When a basis state shared between both modules increases, S(ρ_{ab}) decreases, and mutual information increases. The number of shared basis states between both modules may reflect the degree of connectivity between the genomic modules. However, as each genomic module is complex enough to effect emergence of its own traits, connections should only by parametric for execution.

In the genomic system, a gene shows variable expression levels with respect to state in temporal or sample space. The states |g*ᵢ [symbol] of genes for determining the expression level may be defined in Equation 5. Equation 5 may be replaced with Equation 8 below, with respect to the basis vector of the genome space.

[symbol]

$$\vert {g}_{i}^{*} 〉 = {\sum }_{{j}_{1} = 0}^{1} ⋯ {\sum }_{{j}_{n} = 0}^{1} {j}_{i} {a}_{{ij}_{1}} \vert {j}_{1} 〉 ⊗ ⋯ ⊗ {a}_{{ij}_{n}} \vert {j}_{n} 〉 \qquad [ Equation 8 ]$$

Equation 8 indicates that the expression level of any one gene in the genomic system may be changed dependent on the pattern of interactions with all genes in the genome.

For prokaryotic organisms, all aᵢⱼ of gene i except for aᵢⱼ_{ᵢ} are 0 (zero) because there is no interaction within a genome. According to Equation 8, the active state |g*ᵢ [symbol] of gene i in prokaryotes becomes a null vector. The null vector means that there is no genome space. Therefore, the genome of prokaryotes is subordinate to proteome. Hence, the expression levels of gene is only determined by the active state of itself as a scalar.

However, in the eukaryotic genome, aᵢⱼ of gene i have a non-zero value. Thus, |g*ᵢ [symbol] become multilinear depending on the active state of the whole genome, and is located in the genome space. The same expression level of a gene can have different meanings with respect to the state of the whole genome. Accordingly, for eukaryotic organisms, the entropy S(ρ) of the genomic module indicates functional integrities and activities of genes and their interrelation.

The real space and the genome space are essentially different from each other. The genome space is a 2ⁿ-dimensional space in which the basis state of the genome is defined as a unit vector. The real space is a 3-dimensional space of the real world in which chemical reactions such as the generation of a specific protein occurs in living things through gene activation. It is impossible to directly approach the genome space in order to find out the activity of the genome. Therefore, there is a need for a method for transforming the genome space into a sample space of gene expression. The sample space is an m-dimensional space in which each sample is defined as a unit vector.

A high-throughput technique such as cDNA microarray is capable of measuring gene expression levels of several thousands of genes simultaneously. Since mRNA conveys information from the genome space to the real space, these methods enable us to look into the genome space. A measurement of the gene expression may be a process of mapping the states of the genome from the genome space to the sample space. High-throughput gene expression measurements from m samples transforms information loaded on mRNA in a genome space into a sample space of m-dimensions. A transformation matrix [symbol] may be used to transform the density matrix ρ of the genome space into the density matrix ρ′ of the sample space. The transformation process is shown in Equation 9 below.

$${{ρ}^{′}}_{m \times m} = {ℳ}_{m \times {2}^{n}} {ρ}_{{2}^{n} \times {2}^{n}} {{ℳ}^{+}}_{{2}^{n} \times m} , where {ℳℳ}^{+} = I and {ℳ}^{+} ℳ = I . \qquad [ Equation 9 ]$$

[symbol] ⁺ is a pseudo inverse matrix of [symbol] . The mixed state |g′ᵢ [symbol] of genes in the sample space is given as Equation 10 below.

[symbol]

|g′ᵢ [symbol] = [symbol] |gᵢ [symbol] [Equation 10]

[symbol]

In order to determine the state of the genome or genes directly from the measured expression level |g′ᵢ [symbol] , [symbol] is required. Many factors can affect the transformation matrix, as measurement of gene expression levels can include selection of samples in a temporal or sample space, measuring methods, data) treatments, and so on. Accordingly, |g′ᵢ [symbol] depends on experimental conditions or environments. Therefore, gene expression data is prone to be inconsistent in principle.

Equations 7, 9, and 10 can be summarized in that the probability of a genomic module, represented by the density matrix ρ, contributing to expression of gene i in a genome space. The probability [symbol] gᵢ|ρ|gᵢ [symbol] in the genome space is equal to the probability [symbol] g′ᵢ|ρ′|g′ᵢ [symbol] in any sample place. Furthermore, by unitary transformation, the entropy S(ρ) of the genome space is equal to the entropy S(ρ′) of the sample space. This proves that the above-mentioned probability and entropy are the only parameters unaffected by measurement conditions that cause deviation of gene expression levels. While it is theoretically impossible to obtain the transformation matrix [symbol] for measuring eukaryotic genomes, entropy and probability can be calculated without considering the characteristics of measurement process.

[symbol]

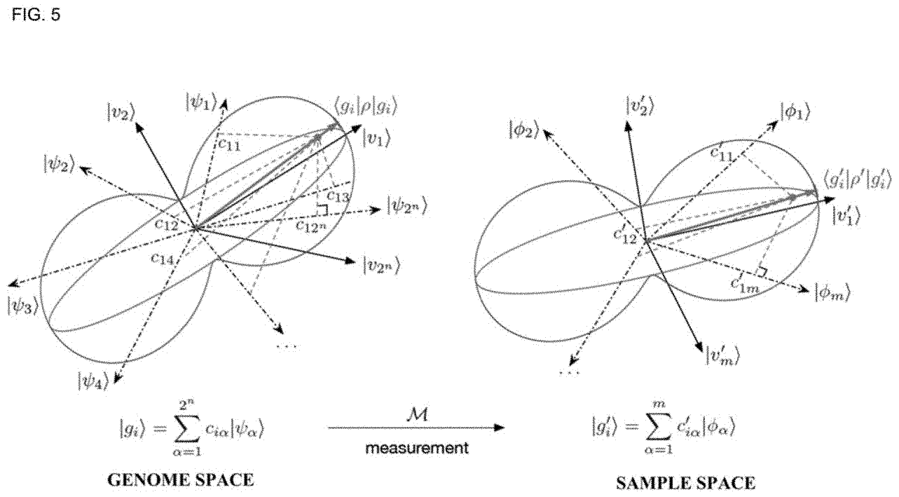

FIG. 5 illustrates an example of a density matrix and a probability of a gene in a genome space and a sample space. In this example, the density matrix ρ and the genetic state |gᵢ [symbol] of genes in the 2ⁿ-dimensional genome space with a basis vector |ψ [symbol] are transformed into ρ′ and |g′ᵢ [symbol] in the m-dimensional sample space with a basis vector |ϕ [symbol] . Here, since [symbol] gᵢ|ρ|gᵢ [symbol] = [symbol] g′ᵢ|ρ′|g′ᵢ [symbol] , the probability of a gene in a genomic module is not affected by the transformation from the genome space into the sample space.

When the vectors of all the genes in the sample space has the same direction, the entropy is equal to 0 (zero). This means that the elliptic density matrix is a straight line that is consistent with the first eigenvector. When the density matrix becomes a perfect circle (or sphere) because the probability of a gene is the same with respect to all the eigenvectors, the entropy has the maximum value.

[symbol] An example of construction for genomic module networks using the real gene expression data will be described below. A tumor can be considered a small independent system that live off the huge host system. Accordingly, the genomic module network can be constructed using genetic information of tumors.

We have used gene expression datasets for constructing genomic module networks. The gene expression datasets include gene expression datasets for cancer tissues and normal tissues.

Gene expression datasets for six primary cancer tissues can includes breast invasive carcinoma (BRCA), colon adenocarcinoma (COAD), rectum adenocarcinoma (READ), lung adenocarcinoma (LUAD), lung squamous cell carcinoma (LUSC), and ovarian serous cystadenocarcinoma (OV). And gene expression datasets for two types of normal tissues can include normal breast tissue (BRNO) and normal colon tissue (CONO). Further, mixture datasets can be used. The mixture dataset can include any combination of six types of cancer tissue data (X6CA), any combination of two types of normal tissue data (X2NO), and any combination of six types of cancer tissue data and two types of normal tissue data (X6C2N) were used. BRCA and other gene expression datasets are obtained by the Cancer Genome Atlas (TCGA) measuring the gene expression level from corresponding tissues. In order to reduce computation time, 36 samples were randomly selected from each dataset. And genomic modules were isolated based on the datasets of samples.

A process of isolating (modularization) the genomic modules based on gene expression datasets will be described below. The density matrices of n modules that are completely independent of one another are represented by ρ₁, . . . ρₙ. Since each space is a Hilbert space, the density matrix of the whole space is equal to ρ=ρ₁⊗ . . . ⊗ρₙ. Accordingly, the whole entropy is equal to the sum of the entropies of the modules. That is

$$S ( ρ ) = S ( {ρ}_{1} ⊗ ⋯ ⊗ {ρ}_{n} ) = {\sum }_{i = 1}^{N} S ( {ρ}_{i} ) .$$

However, when the n modules are not independent (i.e., there is connectivity between the modules), the whole entropy is smaller than the sum of the entropies of the modules. That is,

$$S ( ρ ) < {\sum }_{i = 1}^{n} S ( {ρ}_{i} ) .$$

Any one module may affect another module with exchanging information each other. A module is a collection of genes, and the same gene can be included in different modules. Accordingly, it is difficult for each module to act perfectly independently in the genomic module network. Therefore, modularization may be a process of grouping modules under the condition by minimizing difference between the whole entropy and the sum of the entropies of the modules.

There is no information of participating genes in module, genes participating in several modules at the same time and actual number of modules activated in the genome. Accordingly, it is difficult to find a combination of modules. To solve this problem, modules may be estimated base on local optimal points of true modules.

The estimated module(s) close to the true module could be determined by preventing a transition to other local optimal points.

The estimation modules which the outer boundary partially overlapped corresponds to a domain which includes a group of the true modules expressing the phenotype. The domain for regulating a phenotype has a small number of channels for exchanging information with other domains (max-flow min-cut).

Table 1 below shows an example of pseudocode for an algorithm of finding a local optimal point for a genomic module. A method for finding local optimal point comprises classifying genes into arbitrary sets, removing the genes one by one from each set (module), and lowering the entropy to a target value. Since the found local optimal point is actually inside the module, the target entropy is set to be sufficiently low. In Table 1, “th” corresponds to a threshold value, which is the target value. In Table 1, the backslash indicates an operation of removing a right element from a left set.

TABLE 1

```
Exploration of a local optimum for a genomic module
Require: a gene set Mᵢ = {1ᵢ,...,uᵢ}, where 1ᵢ,...,uᵢ, ϵ {1,...,n}
Require: th
  for gene jᵢ ϵ Mᵢ do
    if S(ρ(Mᵢ \ jᵢ)) > th then
      Mᵢ = Mᵢ \ jᵢ
    end if
  end for
  return Mᵢ
```

The genomic module can be determined based on the local optimal point found by the process described in Table 1. Table 2 below shows an example algorithm for a process of building the genomic module.

TABLE 2

```
Buildup of a genomic module
Require: a gene set Mᵢ = {1ᵢ,...,uᵢ}, where 1ᵢ,...,uᵢ, ϵ {1,...,n}
Require: th
  for gene j ∉ Mᵢ do
    if S(ρ(Mᵢ ∪ j)) ≤ S(ρ(Mᵢ)) and v₁(ρ(Mᵢ ∪ j)) · v₁(ρ(Mᵢ)) ≤ th then
      Mᵢ = Mᵢ ∪ j
    end if
  end for
  return Mᵢ
```

In Table 2 above, an external gene j is added one by one to build up the module under a condition that the entropy is not increased. The fluctuation in the direction of a principal eigenvector vi is limited in order to maintain location of the center of the module in the module build-up process. In Table 2, “th” denotes a threshold value for the fluctuation angle of the principal eigenvector.

Since optimal parameters such as the target value of the entropy in Table 1, the fluctuation angle of the principal eigenvector in Table 2, etc. may vary depending on the properties of the gene expression data. Therefore, there may be a need for a process of determining optimal parameters.

Generally, a low entropy indicates that a system is concentrated on a specific goal represented by the first eigenvector of the density matrix. In the genomic system of eukaryotic cells, genomic modules with low genetic network entropies can be supposed to generate information operating specified phenotypes.

Some of the genes constituting the genomic modules overlap between at least two of modules, since the first eigenvector has a condition with a valueless than or equal to a certain threshold value.

As all tissues producing datasets used in the experiment are composed of many different kinds of cells originating from an endoderm, a mesoderm, and an ectoderm. Accordingly, the expression dataset is acquired from various types of cells. Since the same kind of cells or even an individual cell should perform their own duties, many genes can simultaneously attend to different duties at the tissue level with respect to their own cell. Accordingly, it is difficult to resolve a complex expression profile of a single gene in the tissue.

The genomic modules may be obtained from datasets for the six kinds of primary cancer tissue (BRCA, COAD, READ, LUAD, LUSC, and OV), two kinds of normal tissue (BRNO and CONO), and three random mixtures tissue (X6CA, X2NO, and X6C2N). A module with an extremely low entropy is isolated. The module with the lowest entropy was isolated as, (i) the second modules m2 in most of the tissues and (ii) the first modules m1 in a breast cancer tissue (BRCA), a normal colon tissue (CONO), and an ovarian cancer tissue (OV).

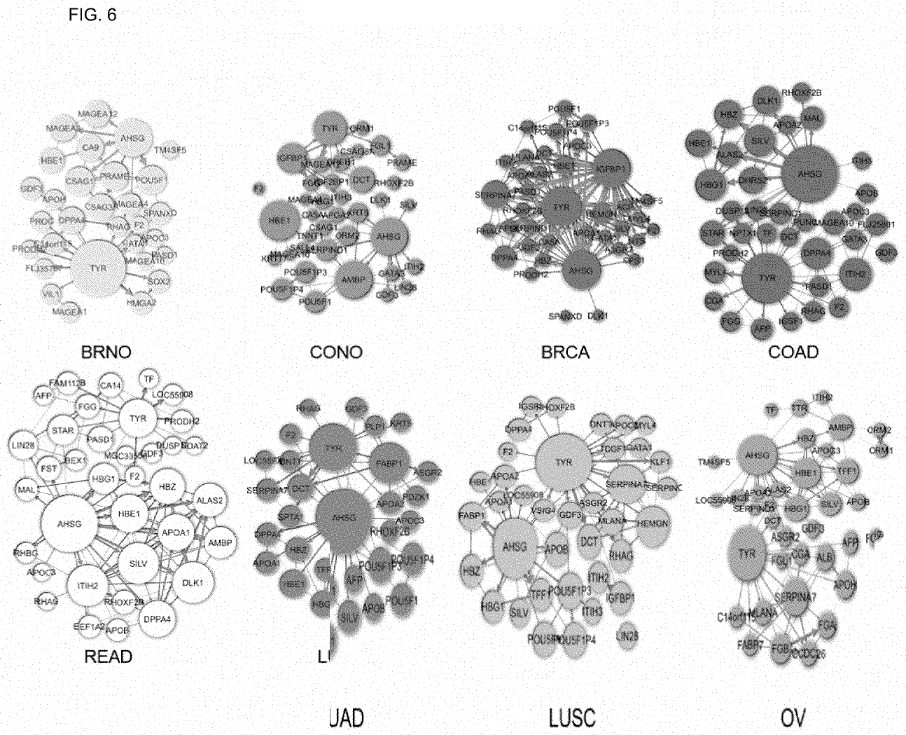

FIG. 6 illustrates an example of genetic networks of a kernel module isolated from each of eight different types of tissue. TOGA dataset for the eight types of tissues are BRNO, CONO, BRCA, COAD, READ, LUAD, LUSC, and OV. Each module consists of a plurality of genes. The genes belonging to each of the modules also construct a certain network. An edge between the genes indicates a channel for transferring or exchanging certain information between the genes. In FIG. 6, the size of each node is proportional to the number of edges between genes. Referring to FIG. 6, genetic modules from different tissues share primary genes. For example, TYR, HBE1, F2, GDF3, and AHSG are shared in all tissues, and the other genes are also shared by more than half of the tissues. Moreover, both TYR and AHSG seem to form axes in the graphical representation of the genetic networks within modules.

A specific module with a very low entropy in any tissue indicates activity in any cell regardless of phenotype and role. Accordingly, the module with a very low entropy may perform certain functions in any kinds of cells, and may be regarded as a core element in the eukaryotic genomic system. The module having an extremely low entropy in any tissues and including shared genes with other modules is hereinafter referred to as a kernel module. There may be a plurality of kernel modules, and a group of the plurality of kernel modules is hereinafter referred to as a kernel domain. Although the biological functions of proteins produced by genes of genomic kernel modules can have meanings in protein networks, their byproducts such as noncoding RNAs constructing the genomic module are important in initiating the genomic system.

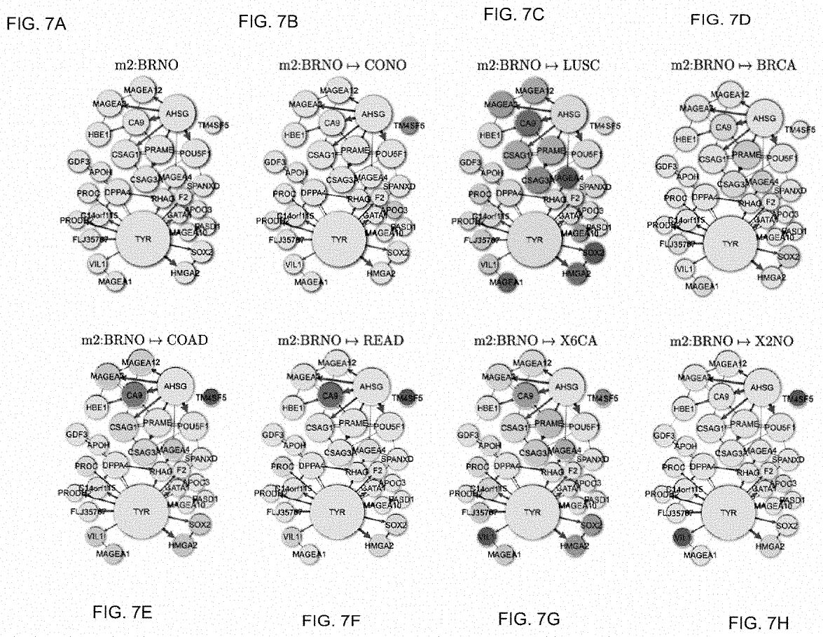

In Experiment kernel modules are mapped between expression datasets from different tissues to confirm whether a kernel module is shared across various tissues. FIGS. 7A-7H illustrate an example of a genetic network of the kernel module of normal breast tissue (BRNO) and the mapping of the kernel module into other tissue types.

FIG. 7A is a primary kernel module of BRNO. FIG. 7B is a result of mapping BRNO to CONO. FIG. 7C is a result of mapping BRNO to LUSC. FIG. 7D is a result of mapping BRNO to BRCA. FIG. 7E is a result of mapping BRNO to COAD. FIG. 7F is a result of mapping BRNO to READ. FIG. 7G is a result of mapping BRNO to X6CA. FIG. 7H is a result of mapping BRNO to X2NO. The nodes of the genetic network are represented in grayscale. A bright node indicates that a log odds ratio (LOR) value of the corresponding gene is close to zero, and a dark node indicates that the LOR value is negative. When the node is brighter, the LOR value is closer to zero. When the node is darker, the absolute value of the negative value increases.

When the kernel of the source dataset is mapped into the target dataset, the mapped entropy should depend on the number of genes included in the kernel domain of the target dataset and the complexity of its interaction pattern. Therefore, if a part of the mapped gene set is not included in the kernel domain or the kernel domain of target dataset has lower complexity than the source, the mapped entropy will increase. When the kernel of BRNO dataset is mapped to other tissue dataset, the entropy increases when a mapped gene is not present in kernel region in other tissue or when complexity of a kernel region of the other tissue is low. When the kernel of BRNO is mapped to CONO, the mapped entropy is 0.091 nats, which is much lower than 0.515 nats, the entropy of a gene randomly selected from the normal colon tissue (CONO). Thus, it can be seen that similarity between kernel modules of two different tissues is considerably high. Referring to FIG. 7B, LOR is close to zero in most genes when the kernel of BRNO is mapped to CONO. When the kernel of BRNO is mapped to tumor data BRCA, COAD, READ, LUAD, LUSC, and OV, the mapped entropy is 0.224 nats to 0.601 nats, which is comparatively high. Accordingly, the high entropy in the mapping from the normal breast tissue (BRNO) to tumor tissues indicates that the kernel region of the tumor has higher directivity than that of the normal tissue. Furthermore, similar results are obtained even when the kernel module of CONO was mapped to other datasets.

There are various domains in a genomic module network, and a domain associated with cell cycle and DNA repair (hereinafter referred to as CCDR) is important in relation to the tumor.

Cell division is an essential process for the development of multicellular organisms from a fertilized egg to somatic cells and the population growth of unicellular organisms. Cell division is elaborately regulated through cell cycle arrest and DNA repair, and regulatory failure may result in abnormal cell growth.

A CCDR domain consists of a plurality of modules in the genome of a normal breast tissue. There are twelve modules that clustered with tightly connected edges and comprise genes known to participate in cell division such as BUB1 mitotic checkpoint serine/threonine kinase. Such modules are also found in other normal tissues datasets CONO and X2NO, and a few modules are found in the tumor tissues datasets.

When twelve CCDR modules in the normal breast tissue are mapped to another normal tissue, the overall values of entropy are as low as those of the primary CCDR modules. In contrast, when the CCDR module is mapped to a tumor dataset, the values of entropy increases as high as the random entropy in the respective dataset.

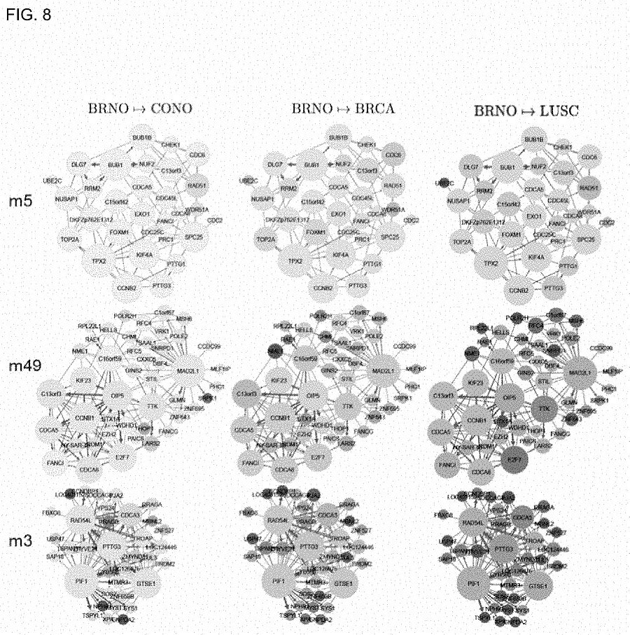

FIG. 8 illustrates an example of mapping a genomic modules on a cell cycle and DNA repair (CCDR) domain of BRNO into other tissue type. FIG. 8 shows a genetic network for some modules in the CCDR domain. FIG. 8 shows m3, m41, and m49 among the modules of CCDR. FIG. 8 is a result of mapping the CCDR module of BRNO into CONO, BRCA, and LUSC, respectively. The meaning of colors of the nodes is as described in FIGS. 7A-7H.

FIG. 8 illustrates an example of a genetic network in modules constituting a CCDR domain of a normal breast. The probability of a module contributing to expression of a gene also indicates how the gene is relevant to the module. When the CCDR modules of the normal breast are mapped into the normal colon (CONO), most genes have high probabilities to the module. In contrast, when the CCDR modules are mapped into a cancer dataset, the probability was significantly decreased. When m3, m41, and m49 are mapped into tumor datasets, the value of entropy mostly exceeds 1.0 nats. This implies that the CCDR program in cancer is not only inoperable, resulting in cancer cell proliferation, but also out of control of the kernel domain and out of balance with the cellular events in the parenchyma and stroma.

Results of mapping the CCDR module of the normal breast tissue into other normal tissues and tumor tissues indicate that a normal cells are under the strict control of the CCDR program, and its disintegration allows cells to continue undergoing cell cycles even with DNA damage. The values of entropy when most CCDR modules of BRNO are mapped to LUAD are twofold lower than the values of entropy when CCDR modules of BRNO is mapped to LUSC. This is consistent with previous studies that show LUSC has a faster growth and a higher frequency of mutation probability than LUAD.

[symbol]

Genetic Network

As described above, a genomic module consists of a plurality of genes. Genes included in one module may compose a network for exchanging information. The network in a module is referred to as a genetic network.

The genomic module is the representation of a program unit in a eukaryotic genome. As described above, a module is configured as a specific unit in the whole program for a living organism. Here, the program indicates a process necessary to drive a system of the living organism.

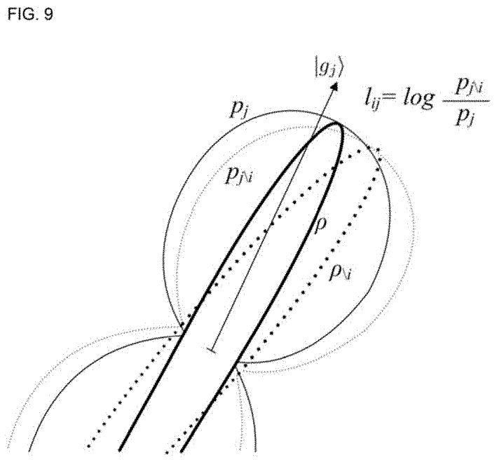

FIG. 9 illustrates an example of a density matrix of a genomic module in a sample space. FIG. 9 shows a density matrix ρ of any module and an expression vector |gⱼ [symbol] of any gene, i.e., gene j included in the module. A thick solid line denotes the density matrix of the module. A normal (thin) solid line denotes a probability locus of a unit vector of the density matrix. A dotted line denotes perturbation due to exclusion of gene i. The perturbation indicates a disturbing motion in a principal force caused by a secondary force in the dynamic system. Since |gⱼ [symbol] is a normalized expression vector of gene j, the probability to the density matrices are on the corresponding locus.

Any module is perturbed by the exclusion of gene i. The density matrix of the perturbed module is called ρ_{\i}. When the module is perturbed by excluding gene i, the density matrix is slightly rotated in the sample space, and its elliptical shape becomes subtly narrower or broader. The probability of gene j in the density matrix is changed from Pⱼ to P_{j\i}. When gene j is strongly connected to gene i, the probability of gene j is significantly decreased by the perturbation.

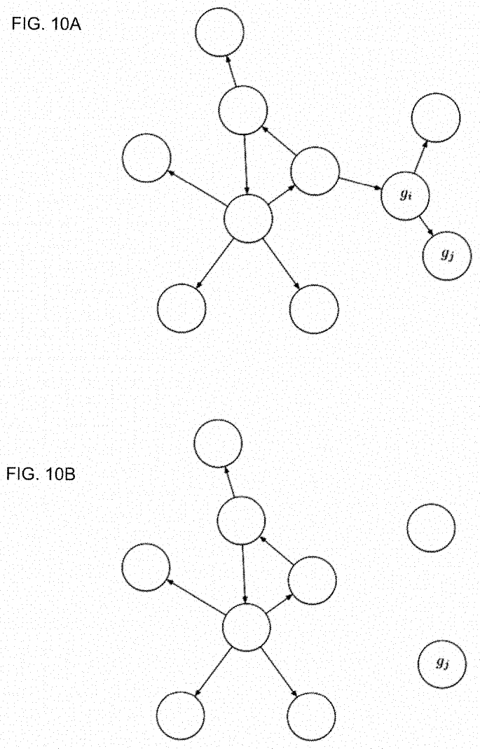

FIGS. 10A and 10B illustrate an example of the change in genetic network of a genomic module by perturbation of exclusion of gene i. FIG. 10A illustrates a genetic network before the exclusion of gene i from any module. FIG. 10B illustrates a genetic network after the execution of gene i. When gene i is removed from the module, gene j connected to only gene i is isolated in the module. Accordingly, the probability of gene j is significantly decreased in the module from which gene i is removed. On the other hand, when gene j is not directly connected to gene i or when gene j is connected to another gene even though gene j is connected to gene i, the probability of gene j is slightly decreased. Accordingly, a connectivity of gene i and gene j can be estimated based on an LOR between the probability of gene j in the module including the gene i and the probability of gene j in the module without gene i.

The odds ratio of the probability may quantitatively describe the influence between two genes. A LOR of the probability is equal to a difference in information content. Equation 11 indicates the degree of probability fluctuation lii of gene j belonging to the same module when gene i is excluded from the module.

$${l}_{ij} = log \frac{{p}_{j \backslash i}}{{p}_{j}} . \qquad [ Equation 11 ]$$

lᵢⱼ can be calculated for all possible gene pairs in the genomic module. When lᵢⱼ for any two genes, i.e., genes i and j exceeds a certain threshold value, it may be estimated that gene i and gene j have strong connectivity. A gene pair having a strong connectivity between them can be depicted by an edge. The genetic network may be configured by computing lᵢⱼ between all the genes present in the genomic module. For example, FIG. 6 illustrates kernel modules for eight tissues, and a genetic network for each module based on the connectivity between gene pair.

Table 3 below shows pseudocode for a process of configuring a genetic network in a module. Briefly, as described above, an LOR is calculated for a gene pair belonging to any module, and an adjacency matrix is generated based on the LOR. LORs between gene i and all other genes are extracted from the adjacency matrix to calculate a quartile, and an internal threshold value thᵢ of gene i is calculated using a cutoff value. A process of connecting an edge between a gene pair which have an LOR greater than or equal to the internal threshold value is performed repeatedly with respect to each gene.

TABLE 3
Reconstruction of genetic network within a genomic module

```
Require: isolated gene set {gᵢ} as a genomic module, where i = 1, ... , n
Require: the log odds ratio matrix L
  for i ≤ n do
    for j ≤ n do
      Probability of gene j for the modular system: pⱼ = (gⱼ|ρ|gⱼ)
      Probability of gene j for the modular system without gene i:
      p_{j\i} = <g_{j\i}|ρ_{\i}|g_{j\i}>
      $Log odds ratio \text{:} {l}_{ij} = log \frac{{p}_{j \backslash i}}{{p}_{j}} \{ {l}_{ij} is an element of the matrix L \}$
    end for
  end for
Require: adjacency matrix A, cutoff value c
  for i ≤ n do
    qᵢ = quartile (l_{i·})
    thᵢ = q_{i,50%} - c · (q_{i,75%} - q_{i,25%})
    for j ≤ n do
      if lᵢⱼ ≤ thᵢ then
        aᵢⱼ = lᵢⱼ {aᵢⱼ is an element of the matrix A}
      else if lᵢⱼ > thᵢ then
        aᵢⱼ = 0
      end if
    end for
  end for
  return A
```

[symbol]

Intermodular Network

As the genetic network is the program operated by genes, the organization of the program is can be represented by an intermodular network. As described above, an intermodular edge is present in a genomic module network. Here, an edge indicates that modules connected by the edge have a certain correlation or connectivity. An edge may be regarded as a channel for transferring or exchanging certain information.

A process of configuring the intermodular network will be described.

For every pair of modules isolated from a dataset, relative entropy are measured for all possible module pairs (module i and module j).

The relative entropy means information gain of module i with respect to module j. The relative entropy may be represented by S(ρᵢ∥ρⱼ)=Tr(ρᵢ ln ρᵢ)−Tr(ρᵢ ln ρⱼ). Here, ρᵢ and ρⱼ indicate density matrices for module i and module j. The relative entropy is always non-negative and non-commutative. i.e., S(ρᵢ∥ρⱼ)≠S(ρⱼ∥ρᵢ). The relative entropy as an indicator of information gain is used to construct the intermodular network. If two density matrices are identical, the relative entropy is zero. When the difference between the two density matrices becomes larger, the relative entropy more increases.

The relative entropy is also used to compare modules in different types of tissues. It is impossible to directly compare the modules separated from the different tissues because the modules have completely different sample spaces. The relative entropy calculated by mapping a module of one tissue to another tissue indicates the difference in density matrix in the same sample space.

When module i has a low information gain with respect to module j, module i and module j are highly correlated. In this case, an intermodular network is constructed by connecting module i and module j by an edge.

In order to increase resolution of the relative entropy at a low level, a negated logarithm may be applied to the relative entropy. The relative entropy to which a log is applied is nlrᵢⱼ=−log(rᵢⱼ), where rᵢⱼ=S(ρᵢ∥ρⱼ), rᵢⱼ>0, and i≠j Table 4 below shows an example of a pseudocode for an algorithm for constructing an intermodular network.

```
TABLE 4
Reconstruction of intermodular network
Require: genomic module set {Mᵢ}, where i = 1, ... , n
Require: the negative log relative entropy matrix N
  for i ≤ n do
    Density matrix of module i: ρᵢ = ρ(Mᵢ)
    for j ≤ n do
      Density matrix of module j: ρⱼ = ρ(Mⱼ)
      Relative entropy: rᵢⱼ = Tr(ρᵢ ln ρᵢ ) - Tr(ρⱼ ln ρⱼ)
      Negative log relative entropy: nlrᵢⱼ = -log(rᵢⱼ) {nlrᵢⱼ is an element of
      the matrix N}
    end for
  end for
Require: adjacency matrix A, cutoff value c
  for i ≤ n do
    qᵢ = quartile(nlrᵢ.)
    thᵢ = q_{i,50%} + c · (q_{i,75%} - q_{i,25%})
    for j ≤ n do
      if nlrᵢⱼ > thᵢ and nlrᵢⱼ > 0 then
        aᵢⱼ = nlrᵢⱼ {aᵢⱼ is an element of the matrix A}
      else if lᵢⱼ ≤ thᵢ or nlrᵢⱼ ≤ 0 then
        aᵢⱼ = 0
      end if
    end for
  end for
  return A
```

In order to determine a correlation of between given modules, i.e., module i to module j, a certain threshold value can be used. When nlrᵢⱼ between module i and module j does not exceed a threshold value, module i and module j are connected by an edge. An appropriate threshold value for determining an intermodular edge should be found with respect to cutoff (C_{f}). For example, as shown in Table 4, the first quartile (Q1), the second quartile (Q2), and the third quartile (Q3) of nlrᵢⱼ may be used.

An information exchange pattern between genomic modules may be represented as an intermodular network. As described before, the relative entropy between the genomic modules measured in the sample space is can be used for determining connection between modules.

Relative entropies may be measured between all possible pairs of genomic modules. The intermodular network may be configured using the adjacency matrix. The adjacency matrix includes relative entropies that do not exceed a threshold value which is determined based on the cutoff. When the intermodular network is constructed from the adjacency matrix, the order of module linkage depends on the type of tissue.

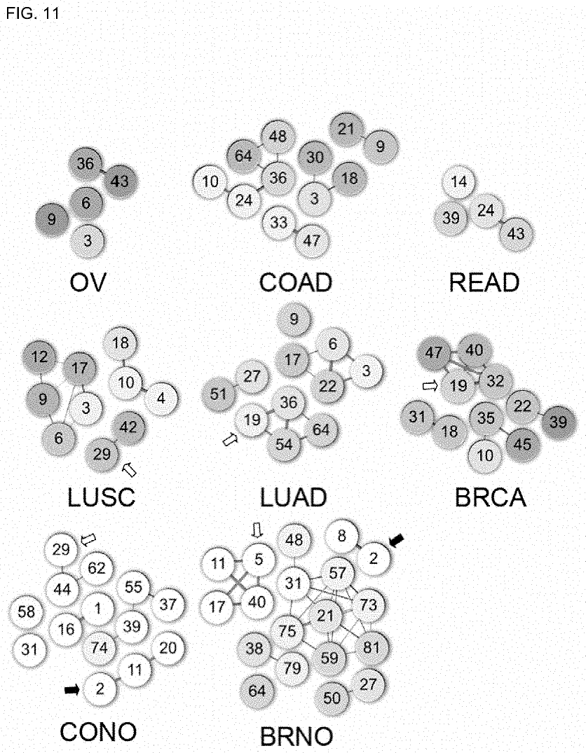

Early-linked modules resemble a seed in a region of in each intermodular network. FIG. 11 illustrates an example of an intermodular network of eight different tissues based on TOGA dataset. A black arrow denotes a seed of a kernel domain, and a white arrow denotes a seed of a CCDR domain. The entropy of the module is represented in grayscale. A brighter color indicates a lower entropy. In FIG. 11, each node represents a single module, and each module is identified by a node number.

For BRNO and CONO datasets, the seed of the kernel domain appears at cutoff (C_{f}) 4.0. However, the first edge in the kernel domain of BRCA does not appear until C_{f} 2.2. The intermodular networks of LUAD, COAD, and READ datasets, shows the first edge of the kernel domain appeared at C_{f} 3.0, C_{f} 2.8, and C_{f} 3.0, respectively. For LUSC and OV, the first edge of the kernel domain appears at C_{f} 1.9. These results suggest that the intermodular networks of a tumor may be different from those of a normal tissue with respect to the kernel domain.

The intermodular network can be reconstructed by varying C_{f} fot TOGA dataset. The total number of edges and the number of edges per module in a normal tissue are larger than those of tumor tissue. This implies that the genomic system of tumor is simpler than that of the normal tissue.

[symbol]

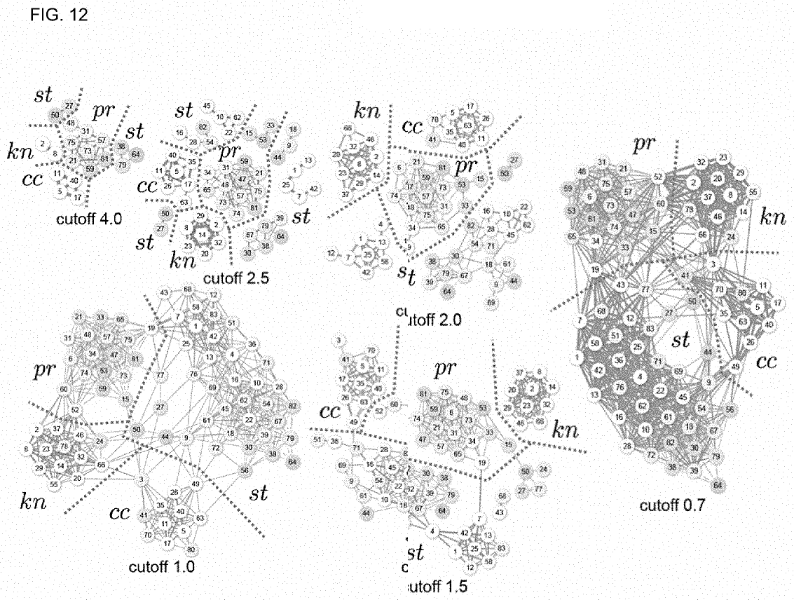

FIG. 12 illustrates an example of an intermodular network of BRNO at various cutoffs. In FIG. 12, kn is a kernel domain, cc is a CCDR domain, pr is a parenchyma domain, and st is a stroma domain. In FIG. 12, each node is depicted with a grayscale value. A brighter color indicates the lower entropy. When cutoff (C_{f}) is lowered, the number of modules having an edge increases, and network disruption is reduced. The intermodular network exhibits complete connections between domains before C_{f} 1.0. The seeds of all the domains kn, cc, pr, and st may appear at C_{f} 4.0.

By mapping modules between gene expression datasets and searching functions of elementary genes from gene ontology, the specific biological function of each region can be inferred. The intermodular network of BRNO configured at C_{f} 1.0 may explain the relationship between domains. The kernel domain kn may control a module pr as the parenchyma function through modules m52 and m60. Module m3 relays an information flow between the kernel domain and the CCDR domain.

A module of the st region may play a role in the stroma function of a normal breast. At C_{f} 4.0, the st region may be divided into two regions. The region including m38, m64, and m79 may be related with adipocytes and the region including m27 and m50 may relay information between the stroma domain and the kernel domain.

The stroma domain st becomes six seeds at C_{f} 2.5 that are suggested to operate angiogenesis, immune function (macrophage), extracellular matrix formation, and adipocytes and to relay between the kernel domain and the CCDR domain.

Functions of domains and modules may be estimated based on intermodular networks determined by C_{f} value.

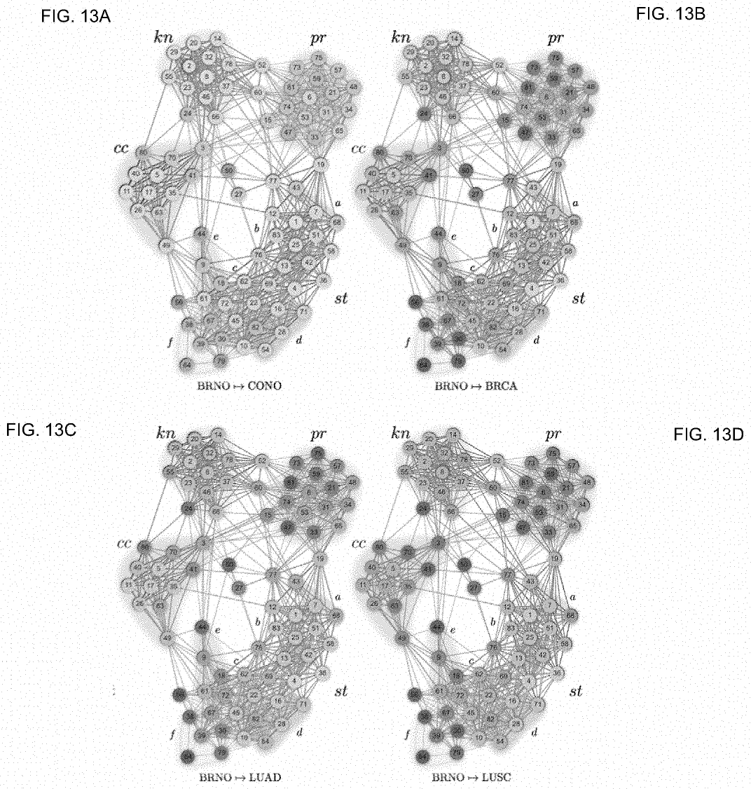

A few modules located in the central area of the intermodular network connect all domains of the genomic system. The a few modules may be considered a kind of meta-program. These suggest that the extracellular matrix and a vasculature are constructed under operating control generated by close communications among stroma module, parenchyma module, and kernel module. The genomic system related to immune function in a normal breast tissue seems to be suppressed by others.

[symbol] FIGS. 13A-13D illustrate an example of an intermodular network of BRNO mapped to other type of tissue. A node color indicates a variation of entropy in the genomic module of BRNO. Each node has a grayscale value. A bright color indicates that the entropy is almost the same in BRNO and other tissues. A dark color indicates that the entropy is higher in other tissues than in BRNO.

FIG. 13A is an example in which BRNO is mapped into CONO, FIG. 13B is an example in which BRNO is mapped into BRCA, FIG. 13C is an example in which BRNO is mapped into LUAD, and FIG. 13D is an example in which BRNO is mapped into LUSC. FIG. 13A is an example of mapping to another normal tissue, and FIGS. 13B to 13D are examples of mapping to a tumor tissue. The intermodular network is constructed based on a domain type and entropy difference between modules in the genomic module network. In FIGS. 13A-13D, f denotes a adipose tissue domain.

Referring to FIG. 13A, when the intermodular network of BRNO was mapped into CONO, most modules in the kernel domain kn shows mapped entropies of 0.091 nats to 0.182 nats similar to those of primary modules in BRNO of 0.017 nats to 0.109 nats. However, the kernel of CONO mapped to BRNO yields mapped entropies of 0.144 nats to 0.289 nats, which is slightly higher than the entropy (of 0.016 nats to 0.043 nats) of CONO itself. This difference may be due to the slightly wider kernel domain of CONO relative to that of BRNO. Accordingly, it may be inferred that colonic tissue is composed of more cell types than a breast tissue. In other domains, a few modules in the parenchyma (pr) and adipose tissue (f) of normal breast reveal mapped entropies that increased somewhat. Accordingly, a large proportion of the biological program of a normal breast tissue should also be active in a normal colon, but the degree of their functional activities may be altered according to parametric inputs from the environments and other modules. Almost all of the mapped modules of CONO to BRNO aside from the kernel domain are much less active than the corresponding primary modules of CONO. This result implies that the genomic system of a normal colon is more complex than that of a normal breast.

FIGS. 13B to 13D show results of mapping the intermodular network of BRNO to BRCA, LUAD, and LUSC, which are tumor datasets. Although the type of cancer differs, the distribution patterns of entropy in mapped intermodular network are similar for all three tumor datasets.

Broadly, the entire parenchyma domain pr of BRNO seems to be completely collapsed, where the entropies of all modules mapped into tumor datasets are 0.890 nats to 1.493 nats. These entropies are much higher than an original entropy 0.109 nats in BRNO and a mapped entropy of 0.263 nats when BRNO is mapped into CONO.

The CCDR domain, whose mapped entropies ranged from 0.754 nats to 1.507 nats, shows different breakage patterns for different tumor types.

In particular, meta-modules for connecting domains are deactivated in a tumor tissue. The meta-module can refer to a module for serving to connect different domains.

The high mapped entropies of module m3, 0.795 nats to 1.407 nats, indicates disintegration of the CCDR domain for intermodular networks mapped into LUSC.

The kernel, CCDR, and parenchyma domains of the genomic system can send parametric information to the stroma domain for controlling extracellular matrix formation including formation of angiogenesis (c), immune function (d), and adipose tissue (f). Regions a and e for connecting the parenchyma domain pr, the kernel domain kn, and the CCDR domain cc to the stroma domain st are very weakened in a tumor tissue. This implies that it is difficult in the tumor tissue that the stroma domain communicates with the other domains. That is, the stroma domain cannot transfer information to other domains which are related with a certain function. This is consistent with uncontrolled stromal construction in a tumor tissue.

[symbol]

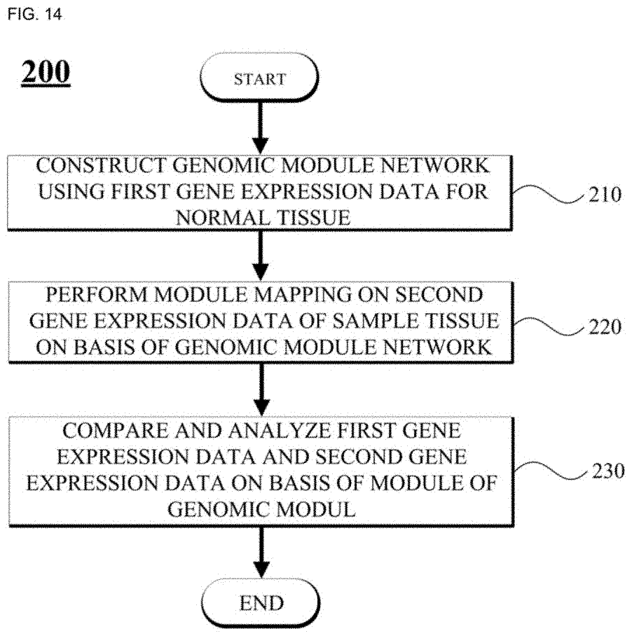

FIG. 14 illustrates an example flowchart of a sample data analysis method based on a genomic module network. A computer apparatus uses two types of gene expression data. First type of data is gene expression data for a normal tissue which is a basic data for constructing a genomic module network (hereinafter referred to as first gene expression data). Second type of data is gene expression data for a sample tissue which will be analyzed (hereinafter referred to as second gene expression data). The type of the sample tissue is the same of the normal tissue. The sample tissue may include a tumor patient's tissue.

The computer apparatus constructs a genomic module network based on the first gene expression data for the normal tissue as described before (210). The genomic module network is constructed based on the gene expression data for the normal tissue. The genomic module network may be composed of module identifiers, identifiers of genes belonging to a module, connection information between modules, domain identifiers, identifiers of modules constituting a domain, identifiers of genes constituting a domain, connection information (a genetic network) between genes belonging to one module, etc. During constructing the genomic module network, genes included in modules, connectivity between modules and connectivity between the genes belonging to the modules are being determined.

The computer apparatus performs module mapping for the second gene expression data of the sample tissue based on the constructed genomic module network (220). The computer apparatus determines that the second gene expression data of the sample tissue belong to which module of the constructed genomic module network by using identifiers of genes.

The computer apparatus compares and analyzes the first gene expression data and the second gene expression data based on the modules of the constructed genomic module network (230). The computer apparatus can analyze the sample tissue based on the genomic module network, a plurality of modules belonging to the genomic module network, or any one module belonging to the genomic module network. Hereinafter, a module which be used for analysis among modules of the genomic module network is referred to as a target module.

The computer apparatus compares data of the first gene expression data belonging to a target module with data of the second gene expression data belonging to the same target module. Thus, the computer apparatus may determine variation of gene expression between the sample tissue and the normal tissue.

[symbol]

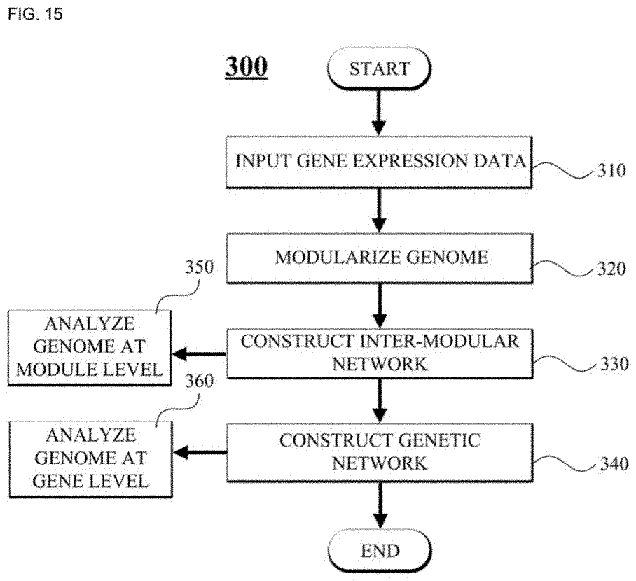

FIG. 15 illustrates an example flowchart of a genomic module network construction process 300.

A computer apparatus receives gene expression data (310). Here, the gene expression data may be gene expression data for normal tissues. The gene expression data may be data extracted from a plurality of samples. The gene expression data is data acquired by utilizing a technique such as cDNA microarray. The computer apparatus modularizes genes into specific modules using the gene expression data (320). This is a process of interpreting the gene expression data and classifying genes constituting a genome into specific modules. The computer apparatus constructs an intermodular network between modules (330). Also, the computer apparatus constructs a genetic network between a genes belonging to a module (340). The genetic network may be constructed after the modularization. The computer apparatus may analyze the intermodular network and analyze the genome at the module level (350). The computer apparatus may analyze a relationship between modules based on the intermodular network. Also, the computer apparatus may also analyze a relationship between different samples by mapping the intermodular network of the samples. As described above, the computer apparatus may analyze the activity of a specific module or a specific domain of a tumor tissue relative to a normal tissue.

Furthermore, the computer apparatus may analyze the genome at the gene level (360). The computer apparatus may analyze a relationship between the genes using the genetic network. Furthermore, the computer apparatus may also analyze a genetic function for a specific sample by mapping the genetic network to different samples. For example, the computer apparatus may perform analysis, such as activation of a specific gene, deactivation of a specific gene, and detection of a gene associated with a tumor. It is possible to find out a gene (marker) associated with a specific disease based on the analysis.

[symbol]

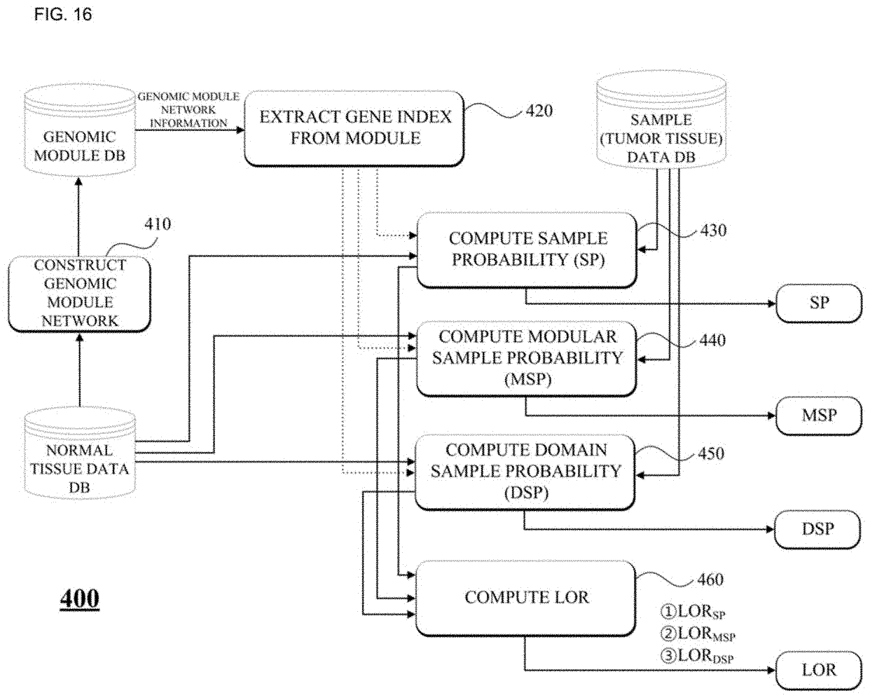

FIG. 16 illustrates an example process of generating an analysis indicator for sample data based on a genomic module network 400. Analysis may be performed through a computer apparatus.

FIG. 16 shows three databases (DBs). A normal tissue data DB stores gene expression information for a plurality of normal tissues (the above-described first gene expression data). A genomic module DB stores information generated after the genomic module network is constructed. A sample data DB stores gene expression information to be analyzed (second gene expression data). The sample data DB may store gene expression information for tumor tissues. The sample data DB may store gene expression information of a plurality of tumor tissues and characteristic information of sample. Hereinafter, it is assumed that the sample data DB may store gene expression information of a tumor patient. The three DBs are depicted separately in FIG. 16, but they may be physically placed in the same storage device.

The computer apparatus generates gene expression vectors of a tumor sample from a tumor tissue data DB. An expression vector is a one-dimensional array generated based on expression data of the whole genome or some genes from a specific sample. The computer apparatus extracts gene expression data of a combination of specific genes from all the samples in the normal tissue data DB, and calculates a density matrix ρ(s) in a gene space by using Equation 6 above. Also, the computer apparatus calculates the probability of a specific sample with respect to the density matrix in the gene space by using Equation 7. The computer apparatus may acquire gene expression data of a tumor tissue sample and generate an expression vector from the acquired gene expression data. The computer apparatus may generate a certain density matrix from the gene expression data of a tumor tissue.

The computer apparatus acquires first gene expression data for normal tissues from the normal tissue data DB. As described above, the computer apparatus constructs a genomic module network on the basis of the first gene expression data (410). The genomic module DB stores information regarding the constructed genomic module network.

The computer apparatus may extract an index of a specific gene from the genomic module DB in order to identify genes belonging to a target module of the genomic module network (420). The genomic module DB provides information regarding which genes constitute a specific module or information regarding which modules constitute a specific domain. Also, the genomic module DB may provide information regarding which module or domain contains a corresponding gene on a gene basis. The genomic module DB may include module identifiers, domain identifiers, gene identifiers, a table matching modules to genes, a table matching domains to modules, a table matching domains to genes, etc.

The computer apparatus compares and analyzes a normal tissue with a tumor tissue by using information provided by the normal tissue data DB, the sample data DB, and the genomic module DB. The computer apparatus may generate various indicators in order to quantify a variation of a tumor tissue relative to a normal tissue. FIG. 16 shows an example of generating the indicators including a sample probability (hereinafter referred to as SP), a modular sample probability (hereinafter referred to as MSP), a domain sample probability (hereinafter referred to as DSP), and a log odds ratio (hereinafter referred to as LOR).

The gene expression data for generating the indicators may be the gene expression data for constructing the genomic module network or a gene expression data originated from a different normal tissue.

The computer apparatus may compute an SP (430). The SP is a value for quantifying the degree of variation of a sample (tumor tissue) relative to normal tissue with respect to all genes included in all genomic modules. The SP indicates the degree of variation of a currently input sample relative to a reference (normal tissue) based on all the genomic modules. The SP is a value obtained by analyzing all the genes included in all the genomic modules. In order to compute the SP, the computer apparatus extracts indices of all genes included in one or more of the genomic modules, determines a density matrix in a normal tissue, and configures an expression vector with a corresponding gene from specific sample data. The SP is represented as a certain probability with respect to sample data. The probability of sample i with respect to a corresponding gene set may be represented by Equation 12 below. The probability of sample i is calculated by using a density matrix P calculated in the gene space defined by corresponding genes using Equation 6 and also by using an expression vector sᵢ composed of expression data of a corresponding gene in sample i.

[symbol]

Pᵢ= [symbol] sᵢ|ρ|sᵢ [symbol] [Equation 12]

[symbol]

Actually, the expression data of genes included in all modules of the normal tissue are reference data for the degree of variation of the genomic system of sample i. Accordingly, the SP may be represented by Equation 13 below. That is, the SP is equal to Pᵢ computed in Equation 13.

$${P}_{i} = P ( {s}_{i} \vert {G}_{M} ) = {σ}_{iM}^{⊤} {ρ}_{M}^{( s )} {σ}_{iM} . \\ {ρ}_{M}^{( s )} = \frac{{G}_{M} {G}_{M}^{⊤}}{t ( {G}_{M} {G}_{M}^{⊤} )} . \\ {σ}_{iM} = \frac{{s}_{Im}}{‖ {s}_{iM} ‖} \qquad [ Equation 13 ]$$

In Equation 13, G_{M} denotes an expression matrix of all gene sets included in one or more of modules for the normal tissue, and s_{iM} denotes an expression vector configured by identifying a corresponding gene in specific sample data sᵢ.

In order to compute the SP, the computer apparatus identifies all the genes belonging to one or more of the genomic modules for the normal tissue, extracts the expression data of the corresponding gene from a normal tissue reference data DB to configure the density matrix, and extracts the expression data of the corresponding gene from tumor tissue reference sample data to configure the expression vector.

The computer apparatus may compute an MSP (440). The MSP refers to a sample probability for each module. While the above-described SP is a sample probability of quantifying the degree of variation relative to the normal tissue with respect to all the genes included in all the genomic modules, the MSP refers to a sample probability calculated with respect to one module. In order to compute the MSP, the computer apparatus extracts indices of genes included in a specific genomic module, determines a density matrix in a normal tissue, and configures an expression vector with a corresponding gene from specific sample data. The MSP refers to the degree of variation of a specific sample with respect to a same specific module in the normal tissue. That is, the MSP is a value for quantifying the degree of variation of the genomic system in the specific sample based on one module. Depending on the disease (e.g., a specific tumor), a large variation may appear in a specific module. Accordingly, the analysis of the MSP is also a meaningful indicator for diagnosing or predicting a disease. Further, as will be described later, the MSP is also used to classify samples in a uniform manner. The MSP may be represented by Equation 14 below.

[symbol]

$$P ( {s}_{i} \vert {G}_{α} ) = {σ}_{i α}^{⊤} {ρ}_{α}^{( s )} {σ}_{i α} . \\ {ρ}_{α}^{( s )} = \frac{{G}_{α} {G}_{α}^{⊤}}{tr ( {G}_{α} {G}_{α}^{⊤} )} . \\ {σ}_{i α} = \frac{{s}_{i α}}{‖ {s}_{i α} ‖} \qquad [ Equation 14 ]$$

In Equation 14, G_{α} denotes an expression matrix of a set of genes included in a specific module α of a normal tissue. s_{iα} (denotes a gene expression vector included in the specific module α in specific sample data That is, the MSP indicates a variation of the genomic system of a specific sample tissue confirmed on the basis of the specific module of the normal tissue. Accordingly, in order to compute the MSP, a genomic module network should be constructed in advance.

The computer apparatus may compute a DSP (450). The genomic module domain, which may be a group of genomic modules having common biological function, consists of adjacent modules in the genomic module network. The DSP refers to a sample probability calculated with respect to all genes included in a specific domain. In order to compute the DSP, the computer apparatus extracts indices of all genes included in one or more of the modules belonging to the specific domain, determines a density matrix from the normal tissue data, and configures an expression vector with a corresponding gene from sample data to be analyzed. The DSP refers to the degree of variation of a sample with respect to a specific genomic module domain of the normal tissue. That is, the DSP is a value for quantifying the degree of variation of the genomic system in the sample based on one domain. The DSP will be described using Equation 14. In Equation 14, G_{α} denotes an expression matrix of a set of genes included in modules belonging to a specific domain α of a normal tissue, and s_{iα} (denotes a gene expression vector configured by extracting data of a corresponding gene from the sample data sᵢ.

The computer apparatus may compute an LOR of a specific gene with respect to a sample probability (460). The LOR is a generalized term that means a log ratio of a probability for the presence or absence of a specific condition. The above-described LOR refers to a degree of a probability fluctuation in a genomic module depending on the presence or absence of a specific gene in one genomic module. The LOR is a value for quantifying connectivity between the genes. The fluctuation in the sample probabilities (SP, MSP, and DSP) depending on the presence or absence of a specific gene in a sample is also a kind of the LOR. That is, the LOR of the specific gene with respect to the sample probability is a value for quantifying the influence of the corresponding gene on the variation of the genomic system in the sample. The LOR is an analysis result considering one gene. The computer apparatus may compute several LOR indicators. (1) LOR_{SP} is a value for quantifying the degree of influence of a specific gene on a sample probability (SP) for all genomic modules in a sample to be analyzed. (2) LOR_{MSP} is a value for quantifying the degree of influence of a specific gene on a sample probability (MSP) for a specific genomic module in a sample to be analyzed. (3) LOR_{DSP} is a value for quantifying the degree of influence of a specific gene on a sample probability (DSP) for a plurality of genomic modules belonging to a specific domain in a sample to be analyzed.

[symbol]

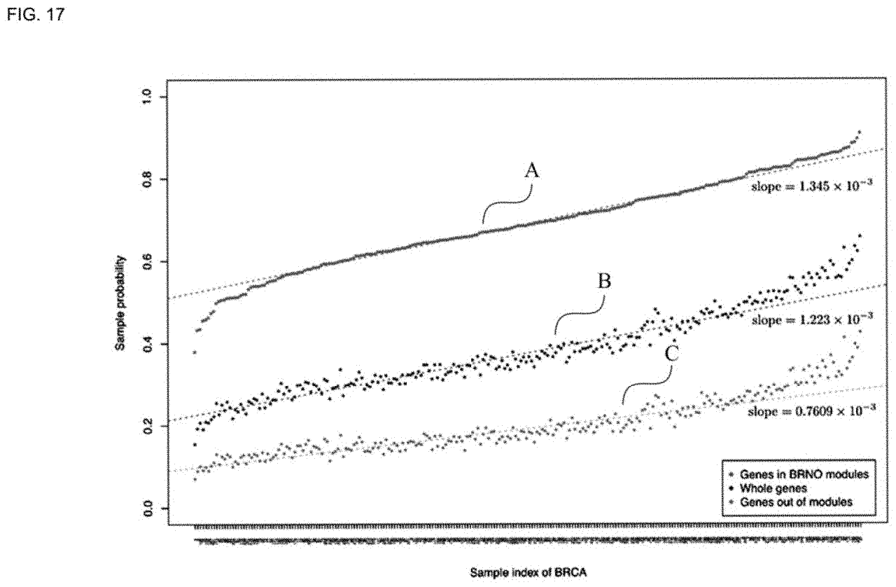

FIG. 17 illustrates an example of sample probabilities (SPs) of a plurality of tumor tissue samples based on a plurality of genomic modules of a normal tissue. In FIG. 17, each dot denotes an SP of a corresponding sample. FIG. 17 shows SPs of 248 breast cancer tissue (BRCA) samples. In FIG. 17, A denotes an SP of each breast cancer sample for all genes included in a normal breast tissue (BRNO) module. This SP is calculated as Equation 13 above. In FIG. 17, B denotes a probability of each breast cancer sample for the whole genes, and C denotes a probability for all genes which do not belong to any modules. As described above, the SP refers to the degree of variation of the total genomic system of the corresponding sample. When a sample has a large variation of the genomic system, the SP is small and is located to the left in the graph of FIG. 17. In FIG. 17, the sample probabilities of the whole genes are low overall than those of all genes in the BRNO module, but have a similar slope each other. Whereas the sample probabilities for all genes which do not belong to any modules have about a half slope of BRNO, which indicates the deviation among the samples is small. The graph of FIG. 17 shows that the genes which do not belong to any modules in each tumor sample do not affect the variation of the genomic system, and the genes belonging to modules affect the variation of the genomic system. FIG. 17 shows that the sample probabilities, i.e., the SPs using the genes included in the genomic module of the normal tissue affect the variation of the genomic system of each tumor sample well.

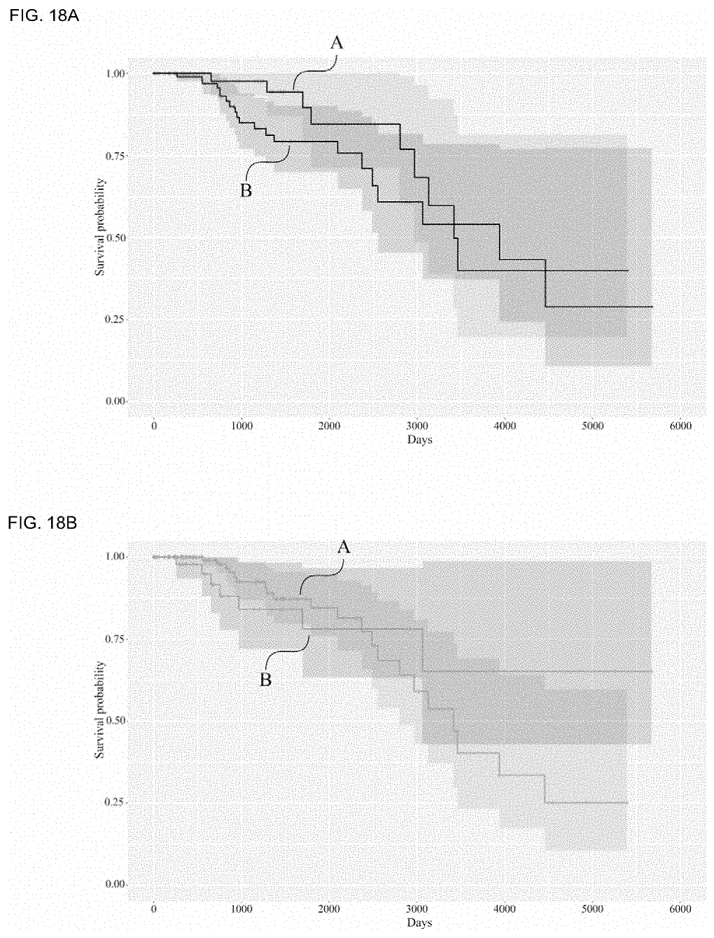

FIGS. 18A and 18B illustrate an example of the survival analysis in tumor sample groups classified based on an SP. FIGS. 18A and 18B are result of Kaplan-Meier survival analysis based on information of patients death and their dates of death for 248 breast cancer patients. FIG. 18A shows calculated SPs for each sample and survival analysis on sample group A in which SPs are greater than or equal to 0.746 and sample group B in which SPs are less than 0.746. The adjacent region of the survival curve means a 95% confidence interval. FIG. 18A shows that the sample group in which SPs are greater than or equal to 0.746 has a significantly higher probability (p-value<0.05) that a patient will survive for about 1,700 days with respect to the sample group in which SPs are less than 0.746. FIG. 18B shows a result of survival analysis on each sample group classified by the presence or absence of expression of an estrogen receptor (ER), which is commonly used as a conventional standard for breast cancer treatment. FIG. 18B shows a result of survival analysis on breast cancer sample group A of ER + (positive) and breast cancer sample group B of ER − (negative). FIG. 18B shows that the ER expression is independent of the survival rate of breast cancer patients. FIGS. 18A and 18B suggests that it is possible to predict the survival time of the patient by calculating an SP of a patient sample based on a genomic module.

[symbol]

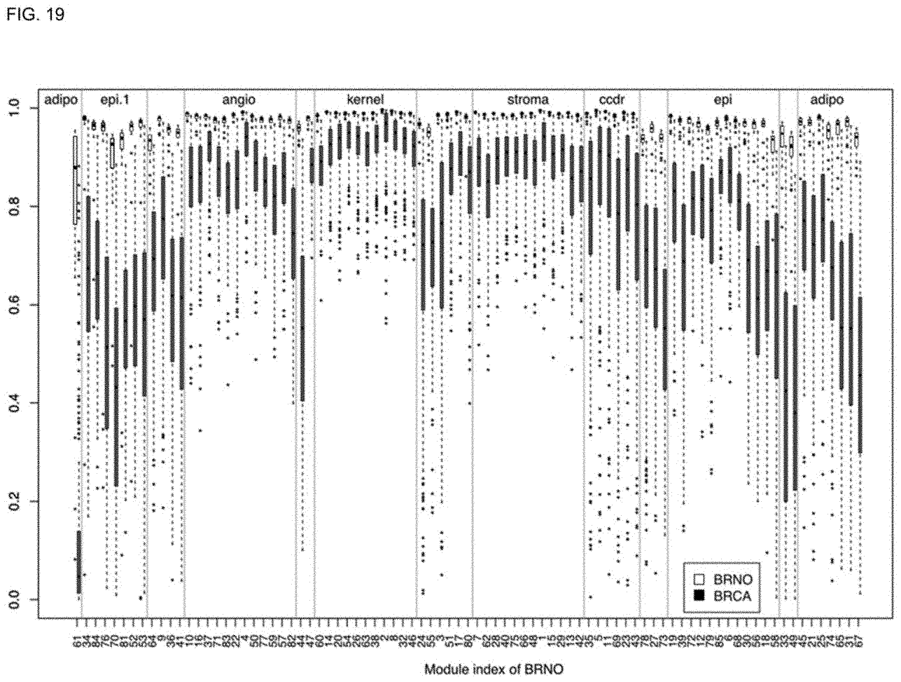

FIG. 19 illustrates an example of modular sample probabilities (MSPs) of a plurality of tissue samples based on each genomic module of a normal tissue. FIG. 19 shows MSPs of a breast cancer sample (BRCA) and a normal breast tissue sample (BRNO) for each genomic module of a normal breast tissue as a box plot. In FIG. 19, for each module, black blocks refers to MSPs of 248 breast cancer samples, and white blocks refers to MSPs of 28 normal breast tissue samples. In FIG. 19, a horizontal axis corresponds to the genomic modules of the normal breast tissue classified on a domain basis. In FIG. 19, a sample having a larger variation for a specific module has a low MSP, and is placed at a lower portion of the plot. In FIG. 19, compared to the MSP of the normal breast sample, the MSP of the breast cancer sample has a large deviation between samples and is overall low. This is represented by the length of the box and the location on the vertical axis. In FIG. 19, the MSPs of the breast cancer samples are very low in domains associated with parenchyma formation (epi, epi.1) and adipose tissue formation (adipo) and in a meta-module connecting the domains, and also the MSPs are highly deviated between samples. Also, the MSP of the breast cancer sample exhibits a meaningful difference in domains associated with angiogenesis (angio), kernel, stroma formation (stroma), and cell cycle and DNA repair (ccdr).

[symbol]

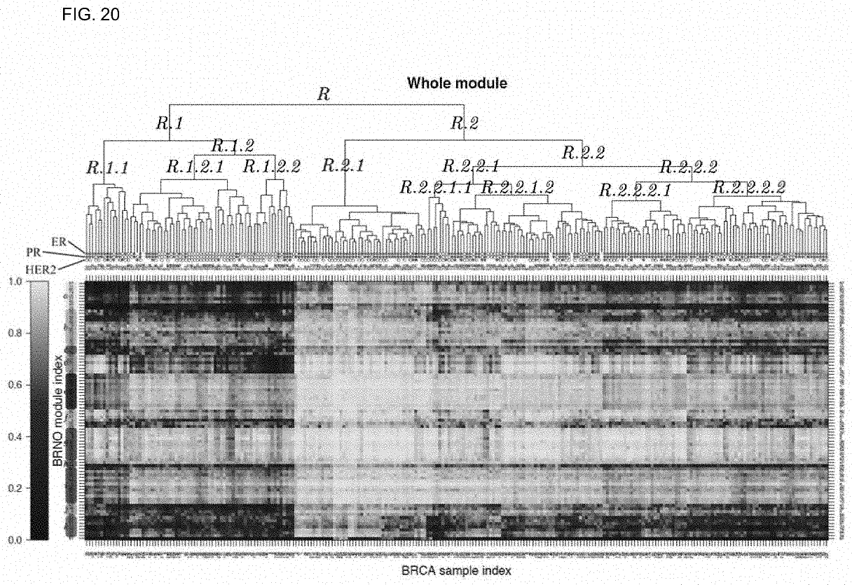

A computer apparatus may classify samples of tumor tissue reference data using sample probabilities. FIG. 20 illustrates an example of tumor tissue samples classification based on an MSPs. FIG. 20 is an example for MSPs of 248 breast cancer patients' tissue samples (BRCA) with respect to 85 genomic modules from a normal breast tissue (BRNO). And FIG. 20 show classified samples based on MSPs. FIG. 20 is an example for hierarchical clustering on MSPs of breast tumor samples with respect to a normal breast tissue. The sample may be classified based on only some or all of the MSPs for the genomic module of the normal tissue. Further, the sample may be classified by DSPs instead of the MSPs.

A heat map at a lower portion of FIG. 20 shows a result of calculating an MSP for each sample with respect to each module. The horizontal axis of the heat map at the lower portion of FIG. 20 corresponds to breast cancer samples aligned through the clustering, and the vertical axis corresponds to classification of the genomic modules of the normal breast tissue by domain, which is consistent with the order of modules of the horizontal axis of FIG. 19. A dendrogram at an upper portion of FIG. 20 shows a result of clustering on breast cancer samples on the basis of all MSPs of 85 modules of the normal breast tissue. In FIG. 20, “R” refer to sample groups according to hierarchical classification. In consideration of a deviation in number of samples, the classification was made to eight breast cancer sample groups R.1.1, R.1.2.1, R.1.2.2, R.2.1, R.2.2.1.1, R.2.2.1.2, R.2.2.2.1, and R.2.2.2.2 in total. The heat map in FIG. 20 shows that R.2.1 is composed of samples with higher MSPs than those of the other sample groups.

Three columns consisting of dots shown at a lower portion of the dendrogram of FIG. 20 indicate whether an estrogen receptor (ER) is expressed, whether a progesterone receptor (PR) is expressed, and whether an HER2 receptor is expressed. State of expression for the receptors is information used for a conventional breast cancer diagnosis and medication policy, and is determined from sample information provided together with breast cancer gene expression data. A red dot indicates that a corresponding receptor is expressed in a corresponding sample, and a white dot indicates that a corresponding receptor is not expressed in a corresponding sample. In FIG. 20, the sample clustering based on MSPs shows a result highly associated with a tumor's characteristic and progression. For example, ample groups R.1.2.1 and R.1.2.2 have much more samples of triple negative (ER, PR, and HER2 are negative) than the other sample groups. In conventional breast cancer diagnosis, a patient is classified as the highest risk when there are triple negative samples, and is classified as a relatively low risk when an ER and a PR are positive.

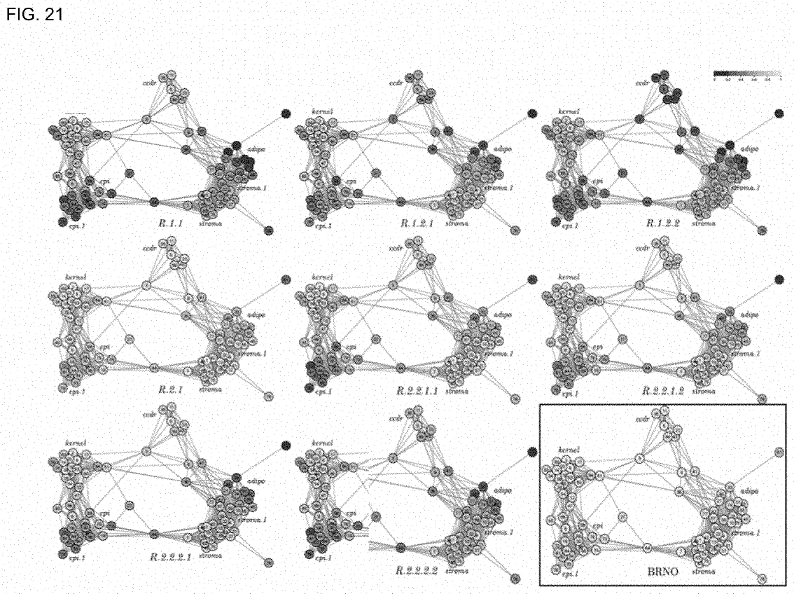

FIG. 21 illustrates an example of an average MSP for each tumor sample group depicted on an intermodular network of a normal tissue. FIG. 21 shows an average MSP for each module of eight breast cancer sample groups classified in FIG. 20 with respect to the genomic module network of the normal breast tissue. The genomic module network at the lower right corner of FIG. 21 shows the average of MSPs for each module of all samples of the normal breast tissue (BRNO). In FIG. 21, each module of the genomic module network is depicted in a brighter color as the average MSP is closer to 1, and is depicted in a darker color as the average MSP is closer to zero. FIG. 21 shows main modules and domains have variation in a genomic system in each breast cancer sample group. FIG. 21 indicates that a main domain where a variation occurs mainly differs in relation with the sample groups. For example, in sample group R.1.2.2, the module variation of the CCDR domain was severe. As described above, the CCDR domain is a domain associated with cell cycle and DNA repair. Accordingly, when a specific patient's sample belongs to R.1.2.2, a medical treatment for targeting cell cycle regulation is expected to be ineffective. This is because a mechanism associated with the cell cycle is very likely not to work properly. Also, sample group R.2.1 exhibits MSPs almost similar to those of a normal breast tissue in all modules except module #61. Accordingly, a patient belonging to R.2.1 is relatively normal and is on early stage of breast cancer. The MSPs and the sample classification through the MSPs may be useful for patient-specific treatment. Furthermore, it can be seen that the classification of modules and domains of a genomic module network is expected as a meaningful technique.

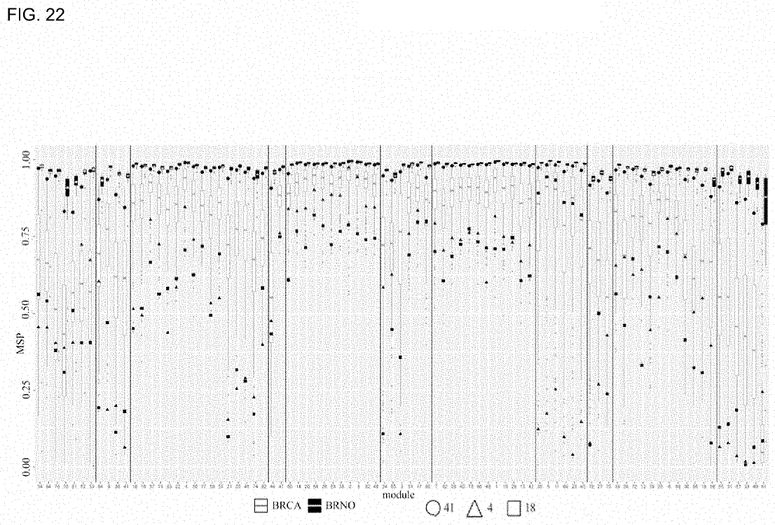

FIG. 22 illustrates an example of MSPs of typical tumor sample belonging to different tumor sample group. FIG. 22 is an example showing the MSP of a sample belonging to a specific breast cancer sample group, depicted as a dot plot on the box plot of FIG. 19. FIG. 22 shows MSPs of sample #41 (a circular point) belonging to sample group R.2.1 which is closest to a normal breast tissue, sample #4 (a triangular point) belonging to sample group R.1.2.2 which is most malignant, and sample #18 (a quadrangular point) belonging to sample group R.1.1 which is also malignant. In FIG. 22, the MSP of sample #41 is very similar to the MSP (a black box) of the normal breast tissue (BRNO) in most modules. On the contrary, the MSPs of sample #4 and sample #18 have low values less than or equal to the first quartile of the MSP (a white box) of the breast cancer tissue (BRCA) in most modules. Sample #18 has an MSP greater than or equal to the median value only in the CCDR domain. Accordingly, it may be estimated that medication for cell cycle regulation is effective for patient #18, unlike patient #4.

[symbol]

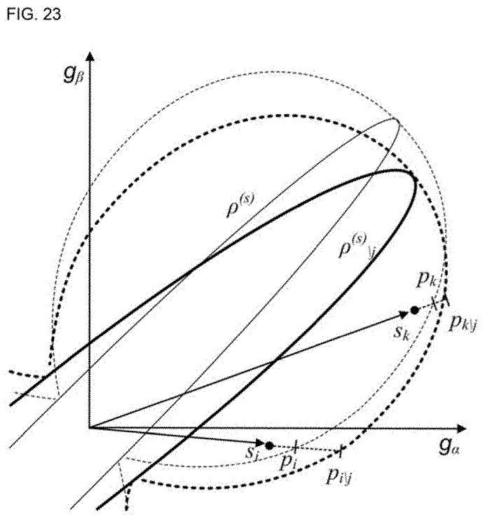

FIG. 23 illustrates an example of a density matrix and probability locus of a genomic module in a gene space. FIG. 23 depicts values for LOR computation. In FIG. 23, an ellipse with a normal (thin) solid line denotes a density matrix ρ(s) for a corresponding gene group, and an ellipse with a thick solid line denotes a density matrix ρ(s)\j when gene j is excluded from a corresponding gene group. In FIG. 23, a dotted line denotes a sample probability locus for each density matrix. An LOR may be represented by Equation 15 below. The LOR may be computed for a specific gene belonging to a specific module.

[symbol]

$${r}_{ij} = - log \frac{{P}_{i \backslash j}}{{P}_{i}} \qquad [ Equation 15 ]$$

The LOR is a value for quantifying the influence of gene j on the sample probability SP, MSP, or DSP in sample data sᵢ. The sample probability is a value for quantifying the degree of variation of the genomic system in the sample to be analyzed, and the LOR is a value for quantifying the degree of influence of a specific gene on the sample probability in the corresponding sample. The LOR is negative for a gene facilitating a variation of the genomic system, and is positive for a gene suppressing a variation of the genomic system.

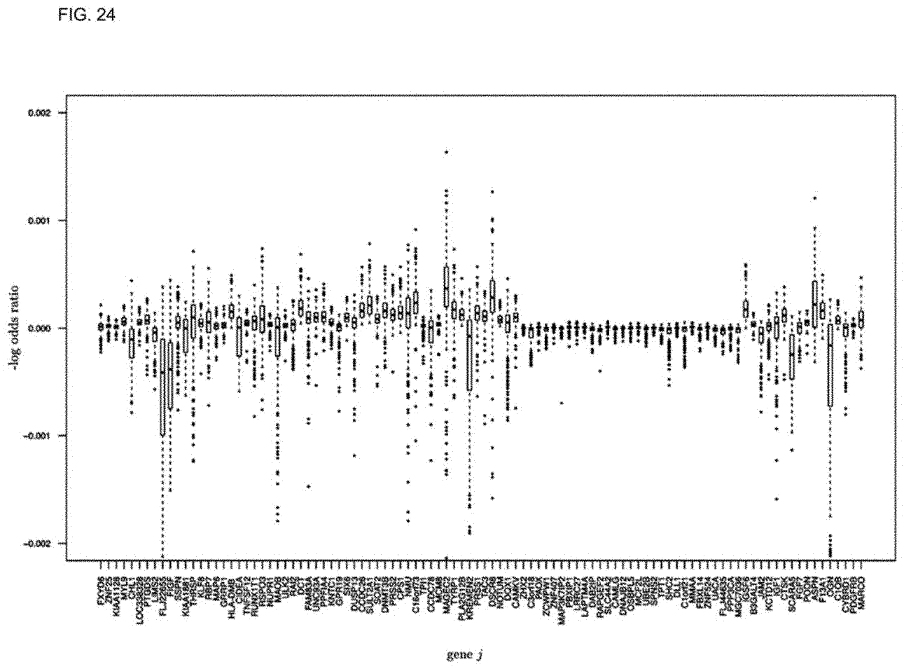

FIG. 24 illustrates an example of box plot of log odd ratios (LORs) versus genes in each tumor sample using a density matrix of a gene group of a normal tissue. FIG. 24 is an example showing an LOR calculated using genes belong to a gene group from each sample of 248 breast cancer tissues and a density matrix of the same gene group from gene expression data of a normal breast tissue. In FIG. 24, the genes have various distributions of LORs in the 248 breast cancer tissues. FIG. 24 shows that the influence of each gene on the variation of the genomic system differs depending on a breast patient. In FIG. 24, a breast cancer sample having an LOR of a specific gene greatly deviated from zero indicates that the sample is highly affected by a corresponding gene in the variation of the genomic system.

In Equation 15, (1) for LOR_{SP}, Pᵢ corresponds to an SP of sample sᵢ, as defined in Equation 13, and P_{i\j} denotes a value obtained by computing an SP of sample sᵢ for a corresponding gene when gene j is excluded from genes belonging to all the genomic modules. (2) For LOR_{MSP}, Pᵢ corresponds to an MSP of sample sᵢ for a specific module α as defined in Equation 14, and P_{i\j} denotes a value obtained by computing an MSP of sample sᵢ for a corresponding gene when gene j is excluded from genes belonging to the specific module α. (3) For LOR_{DSP}, Pᵢ is a value obtained by computing a DSP of sample sᵢ for a gene included in one or more of all modules belonging to a specific domain, and P_{i\j} denotes a value obtained by computing a DSP of sample sᵢ when gene j is excluded from corresponding genes.

A computer apparatus may calculate a sample probability using a density matrix calculated using expression data of a specific combination of genes of a normal tissue and an expression vector consisting of corresponding genes in a sample to be analyzed, and the computer apparatus also calculate an LOR by a sample probability when a specific gene is excluded. Also, since analysis may be performed on a gene belonging to a specific module, the computer apparatus may identify the gene belonging to the specific module with reference to a genomic module DB and then compute an LOR of the corresponding gene.

As described above, an LOR of each gene of a sample can be computed using a normal tissue. Further, the computer apparatus can compute an LOR based on a tumor tissue. In this case, the computer apparatus may determine a density matrix from gene expression data of a tumor tissue, and then configure an expression vector of the sample to be analyzed.

[symbol]

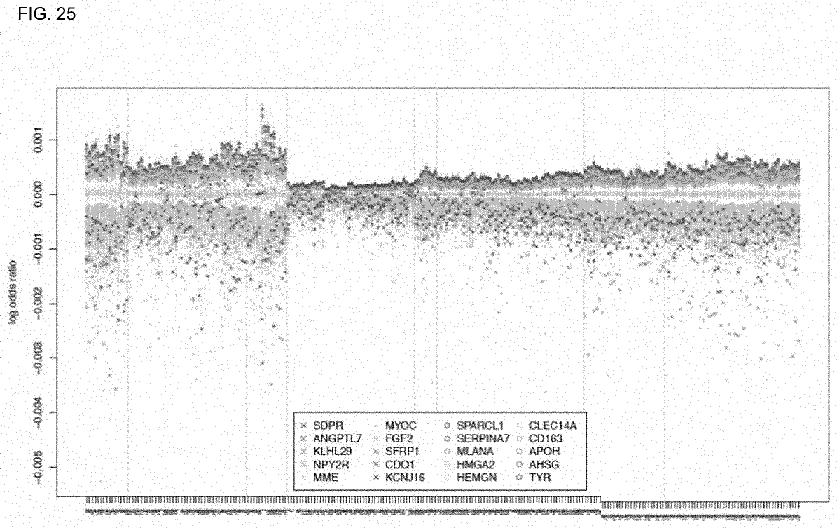

FIG. 25 illustrates an example of dot plot of LORs versus tumor samples calculated for each gene using a density matrix of a genomic module of normal tissue. FIG. 25 is an example of an LOR of a gene calculated for each sample of 248 breast cancer tissue (BRCA) using a module of a normal breast tissue (BRNO). The horizontal axis of FIG. 25 corresponds to breast cancer samples arranged according to the sample classification result using MSPs of FIG. 20. That is, FIG. 25 shows LORs of some genes for each sample of a breast cancer in a dot plot. As an example, FIG. 25 shows LORs of 20 genes having a certain pattern according to a breast cancer patient's samples. In the graph, all genes marked with a mark “x” have negative LORs. That is, the corresponding genes may be regarded as genes facilitating a breast cancer. On the other hand, genes marked with a mark “∘” have positive LORs. Accordingly, the corresponding genes may be regarded as genes suppressing a breast cancer. Thus, LORs of some genes have a certain tendency even in a specific disease such as a tumor. Therefore, the LOR may be useful as a specific indicator for genetic analysis.

[symbol]

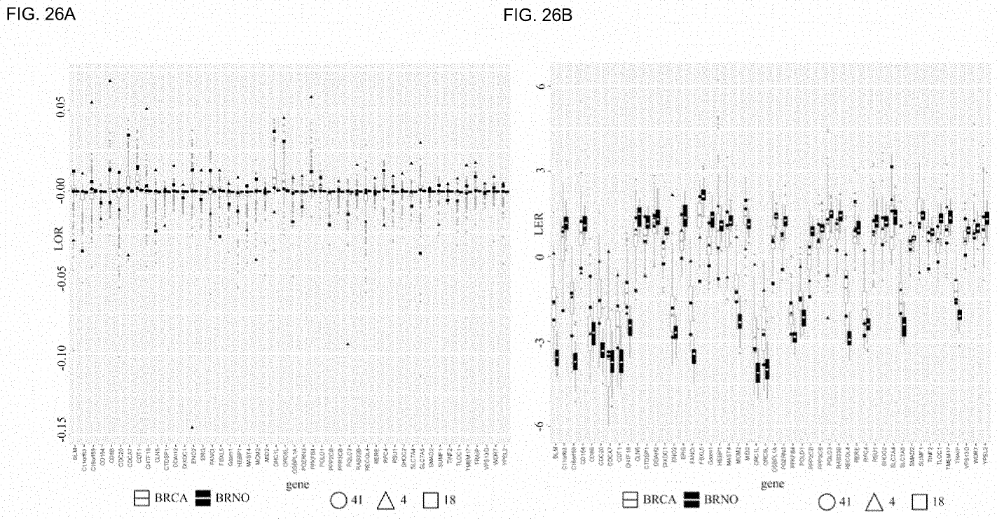

FIGS. 26A and 26B illustrate an example of LORs and log expression ratios (LERs) of genes in each sample of normal and tumor tissues based on a specific genomic module of normal tissue. The LER is a common indicator used in microarray analysis. FIGS. 26A and 26B show graph for LORs and LERs of genes in individual samples of a breast cancer tissue (BRCA) and a normal breast tissue (BRNO). FIGS. 26A and 26B are an example showing LORs and LERs of genes constituting module #9 (module 9# of BRNO) among genomic modules of a normal breast tissue in each tissue sample in a box plot. FIGS. 26A and 26B are result of comparing LOR_{MSP}s and LERs. FIG. 26A shows LORs of genes constituting module #9 of BRNO in individual samples of a normal breast tissue and a breast cancer, and FIG. 26B shows LERs of the genes in the tissue samples.

In FIGS. 26A and 26B, black box data for each gene indicates an LOR_{MSP} and an LER of a corresponding gene in 28 samples of a normal breast tissue (BRNO), and white box data indicates an LOR_{MSP} and an LER of a corresponding gene in 248 samples of a breast cancer (BRCA). FIGS. 26A and 26B show LOR_{MSP}s and LERs of sample #41 (a circular point) belonging to sample group R.2.1 which is closest to the normal breast tissue, sample #4 (a triangular point) belonging to sample group R.1.2.2 which is most malignant, and sample #18 (a quadrangular point) belonging to sample group R.1.1 which is also malignant and has an active function associated with cell cycle, in a dot plot on a box plot of the breast cancer samples. In FIG. 26A, the LOR_{MSP}s of the normal breast tissues are near around zero, and sample #41 of the breast cancer has similar LOR_{MSP}s to those of the normal breast tissue. On the other hand, sample #4 exhibits very large fluctuation around zero in most genes, and sample #18 exhibits a bit smaller fluctuation than sample #4 but significant fluctuation compared to all the samples. Also, for genes having LOR_{MSP}s with large fluctuation in sample #4 and sample #18, the directions of the fluctuation may not be consistent with each other. That is, even among patients with the same kind of tumor, there may be individual differences in the influence of the same gene on the variation of the genomic system. Meanwhile, FIG. 26B shows that LERs do not reflect characteristics of samples at all. The LERs do not indicate that sample #41 of the breast cancer is close to the normal breast tissue and sample #4 and sample #18 are malignant samples in the breast cancer tissue. Accordingly, LORs can indicate the influence of each gene on a sample's characteristics however a conventional method using LERs is not effective.

[symbol]

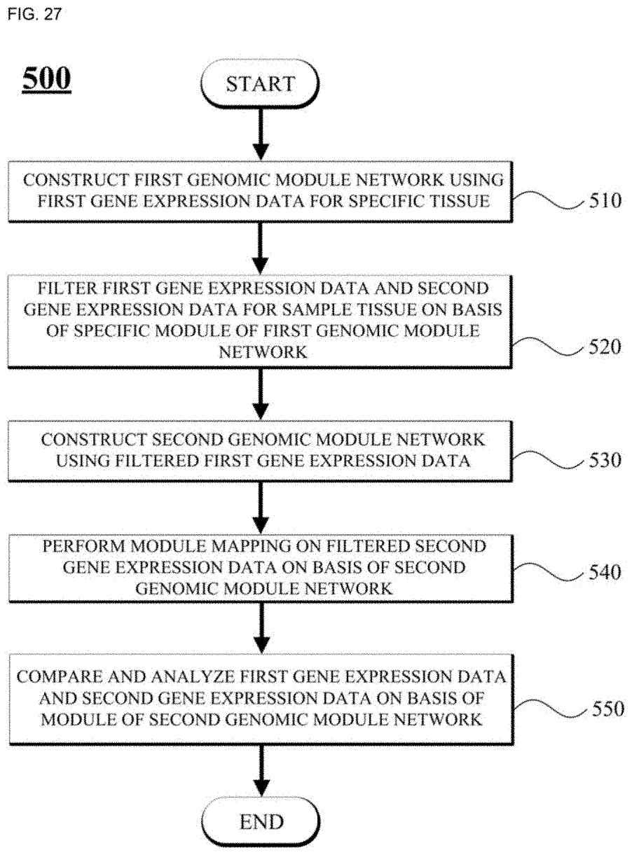

FIG. 27 illustrates an example of flowchart for a sample data analysis method based on genomic module networks constructed with a filtered gene expression dataset.

A computer apparatus uses two types of gene expression data. First type of data is first gene expression data for a specific tissue which is a criterion for constructing a genomic module network. Here, the specific tissue may be a normal tissue or a tumor tissue.

Second type of data is second gene expression data for a sample tissue which will be analyzed. The tissue to be analyzed is a tumor tissue that occurs at the sample position as that of the above-described normal tissue.

The computer apparatus constructs a first genomic module network using the first gene expression data for the normal tissue or the tumor tissue (510). The first genomic module network is constructed on the basis of the gene expression data for the normal tissue or the tumor tissue. The first genomic module network may be composed of module identifiers, identifiers of genes belonging to a module, connection information between modules, domain identifiers, identifiers of modules constituting a domain, identifiers of genes constituting a domain, connection information (a genetic network) between genes belonging to one module, etc. When the genomic module network is constructed, a module is matched to genes belonging to the module, and connectivity with the module and also connectivity with the genes belonging to the module are completed.

The computer apparatus filters the first gene expression data and the second gene expression data of the sample tissue on the basis of a specific module belonging to the first genomic module network (520). The filtering process will be described below in detail. The first genomic module network is composed of a plurality of modules, and any one module has connectivity with at least one module. That is, any one module sends certain information to at least another module. The filtering process corresponds to a process of blocking (filtering) transfer of information to any one module (in some cases, a plurality of modules) that transfers a relatively large amount of information. The specific module to which the information transfer is blocked may be kernel module(s). As described above, the kernel module has a lower entropy than the other modules and is likely to be involved in a various biological function. Meanwhile, a plurality of kernel modules may belong to a kernel domain. In this case, the filtering may be processed with respect to at least one of the kernel modules. For example, the filtering may be processed based on a kernel module with the lowest entropy among the kernel modules.

A specific module to be filtered may be a module with an entropy less than or equal to a certain reference value. The reference value depends on a tissue type to be analyzed, a disease type, a data collection environment, and the like.

The computer apparatus constructs a second genomic module network using the filtered first gene expression data (530). The process of constructing the genomic module network is as described above. The second genomic module network is constructed on the basis of data filtered in a uniform manner.

The computer apparatus performs module mapping on the filtered second gene expression data of the sample tissue based on the constructed second genomic module network (540). That is, the computer apparatus determines to which module each data of the second gene expression data of the sample tissue belongs by using identifiers of genes belonging to a specific module in the second genomic module network.

The computer apparatus compares and analyzes the first gene expression data with the second gene expression data based on the modules of the constructed second genomic module network (550). The computer apparatus analyzes based on all the modules of the genomic module network, a plurality of modules belonging to the genomic module network, or any one module (a target module) belonging to the genomic module network.

The computer apparatus compares first gene expression data belonging to a target module with second gene expression data belonging to the same target module. Thus, the computer apparatus may determine variation of the sample tissue relative to the normal tissue or the tumor tissue. The computer apparatus may analyze the variation of the sample tissue using unfiltered first gene expression data and unfiltered second gene expression data. Also, the computer apparatus may analyze the variation of the sample tissue using filtered first gene expression data and filtered second gene expression data.

[symbol]

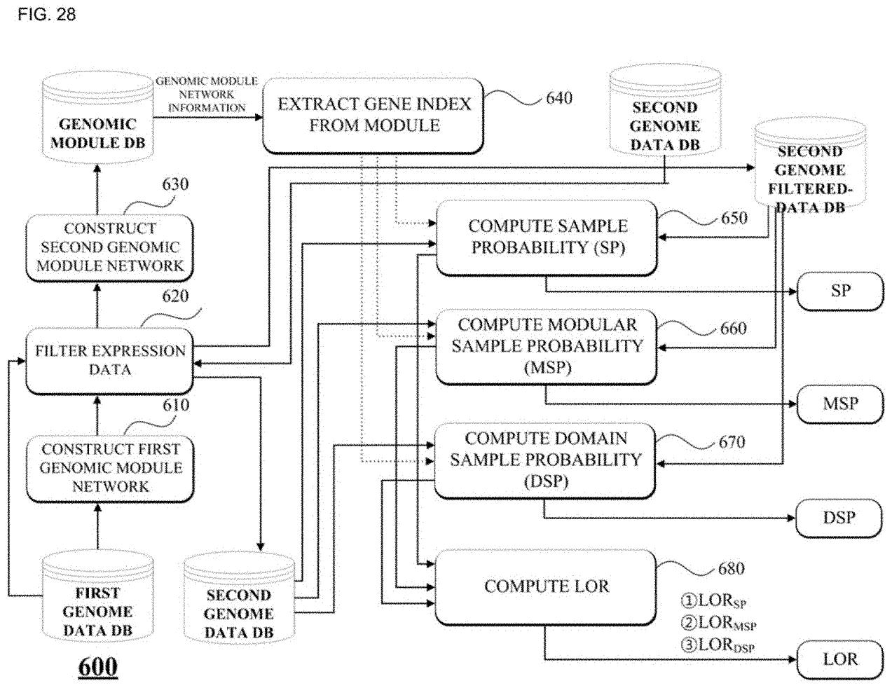

FIG. 28 illustrates an example process of generating analysis indicators for sample data based on genomic module networks constructed with a filtered gene expression dataset 600.

FIG. 28 shows five databases (DBs). A first genomic data DB stores gene expression information for a plurality of normal tissues or tumor tissues. The first genomic data DB stores the above-described first gene expression data. A first genome filtered-data DB stores data obtained by filtering data of the first genomic data DB in a uniform manner.

A genomic module DB stores information generated after the genomic module network is constructed. A second genomic data DB stores gene expression information of an analysis object. The second genomic data DB stores the above-described second gene expression data. The second genomic data DB may store gene expression information for tumor tissues. The second genomic data DB may store gene expression information of a plurality of tumor tissues and individual property information of a corresponding sample. Hereinafter, it is assumed that the second genomic data DB stores gene expression information of a tumor patient. A second genome filtered-data DB stores data obtained by filtering data of the second genomic data DB in a uniform manner.

The five DBs are depicted separately in FIG. 28, but a plurality of DBs may be physically placed in the same storage device.

The computer apparatus acquires first gene expression data for normal tissues or tumor tissues from the first genomic data DB. As described above, the computer apparatus constructs a first genomic module network based on the first gene expression data (610).

The computer apparatus filters the first gene expression data and the second gene expression data based on a specific module belonging to the first genomic module network (620). The first genome filtered-data DB stores the filtered first gene expression data. The second genome filtered-data DB stores the filtered second gene expression data.

The computer apparatus constructs a new second genomic module network based on the filtered first gene expression data (630). The genomic module DB stores information for the constructed second genomic module network.

The computer apparatus may extract an index of a specific gene from the genomic module DB in order to identify genes belonging to a target module of the second genomic module network (640). The genomic module DB may include module identifiers, domain identifiers, gene identifiers, a table matching modules to genes, a table matching domains to modules, a table matching domains to genes, etc.

The computer apparatus analyzes an individual variation of a tumor tissue sample on the basis of the second genomic module network by using information provided from the first genome filtered-data DB, the second genome filtered-data DB, and the genomic module DB. The computer apparatus may generate various indicators in order to quantify a variation of an individual tumor tissue relative to multiple normal tissues or tumor tissues. In this process, the computer apparatus generates an indicator based on the second genomic module network.

The gene expression data for generating the indicators may be gene expression data used to construct the genomic module network or gene expression data extracted from a different tissue. Alternatively, the gene expression data for generating the indicators may be filtered gene expression data or unfiltered gene expression data.

The computer apparatus may compute an SP (650). The SP is a value for quantifying the degree of variation of the genomic system in a sample of an individual tumor patient with respect to all genes included in all genomic modules of a normal tissue or a tumor tissue. The SP represents the degree of variation of a input sample with respect to all genomic modules. The computer apparatus extracts indices of all genes included in one or more of all the genomic modules, determines a density matrix in the normal tissue or tumor tissue, and configures an expression vector with a corresponding gene from specific sample data, thereby computing the SP. The SP is represented as a certain probability with respect to sample data to be analyzed. The probability of sample i with respect to a corresponding gene set may be represented by Equation 12 above. However, the computer apparatus computes the SP based on the second genomic module network. Also, the computer apparatus may compute the SP based on the filtered data.

In order to compute the SP, the computer apparatus identifies all the genes belonging to one or more of the genomic modules of the second genomic module network, extracts the expression data of the corresponding gene from all samples of the first genome filtered-data DB to compute the density matrix, and extracts the expression data of the corresponding gene from the second genome filtered data to configure the expression vector and compute the SP.

The computer apparatus may compute an MSP (660). The MSP refers to a sample probability for each module. While the above-described SP is a sample probability of quantifying the degree of variation from the normal tissue on the basis of all the genes included in all the genomic modules, the MSP refers to a sample probability calculated on the basis of each module. In order to compute the MSP, the computer apparatus extracts indices of genes included in a specific genomic module, determines a density matrix in a normal tissue or a tumor tissue, and configures an expression vector with a corresponding gene from specific sample data. The MSP refers to the degree of variation of a specific sample with respect to a specific module of the normal tissue or the tumor tissue. That is, the MSP is a value for quantifying the degree of variation of the genomic system in the specific sample on a module basis. Depending on the disease (e.g., a specific tumor), a large variation may appear in a specific module. Accordingly, the analysis of the MSP is also a meaningful indicator for diagnosing or predicting a disease. Further, as will be described later, the MSP is also used to classify samples in a uniform manner. The MSP may be represented by Equation 14 above. However, the computer apparatus computes the MSP on the basis of the second genomic module network. Also, the computer apparatus may compute the MSP on the basis of the filtered data.

Meanwhile, the computer apparatus may compute a DSP (670). The genomic module domain, which is a set of genomic modules having similar biological functions, consists of adjacent modules in the genomic module network. The DSP refers to a sample probability calculated on the basis of all genes included in one or more of modules belonging to a specific domain. In order to compute the DSP, the computer apparatus extracts indices of all genes included in one or more of the modules belonging to the specific domain, determines a density matrix from normal tissue data or tumor tissue data, and configures an expression vector with a corresponding gene from sample data to be analyzed. The DSP refers to the degree of variation of a sample to be analyzed with respect to a specific genomic module domain of the normal tissue or the tumor tissue. That is, the DSP is a value for quantifying the degree of variation of the genomic system in the sample to be analyzed on a domain basis. The DSP will be described using Equation 14. In Equation 14, G_{α} denotes an expression matrix of a set of genes included in modules belonging to a specific domain α of a normal tissue or a tumor tissue, and s_{iα} denotes a gene expression vector configured by extracting data of a corresponding gene from sample data sᵢ. However, the computer apparatus computes the DSP based on the second genomic module network. Also, the computer apparatus may compute the DSP based on the filtered data.

The computer apparatus may compute an LOR of a specific gene with respect to a sample probability (680). The LOR refers to a probability fluctuation degree of a genomic module of the other genes depending on the presence or absence of a specific gene in one genomic module and is a value obtained by quantifying connectivity between the genes. Meanwhile, the fluctuation in the sample probabilities (SP, MSP, and DSP) depending on the presence or absence of a specific gene in one sample also corresponds to the LOR. That is, the LOR of the specific gene with respect to the sample probability is a value for quantifying the influence of the corresponding gene on the variation of the genomic system in one sample. The LOR is an analysis result considering one gene. The computer apparatus may compute several LOR indicators. (1) LOR_{SP} is a value for quantifying the degree of influence of a specific gene on a sample probability (SP) for all genomic modules in a sample to be analyzed. (2) LOR_{MSP} is a value for quantifying the degree of influence of a specific gene on a sample probability (MSP) for a specific genomic module in a sample to be analyzed. (3) LOR_{DSP} is a value for quantifying the degree of influence of a specific gene on a sample probability (DSP) for a plurality of genomic modules belonging to a specific domain in a sample to be analyzed. However, the computer apparatus computes the LOR based on the second genomic module network. Also, the computer apparatus may compute the LOR based on the filtered data.

[symbol]

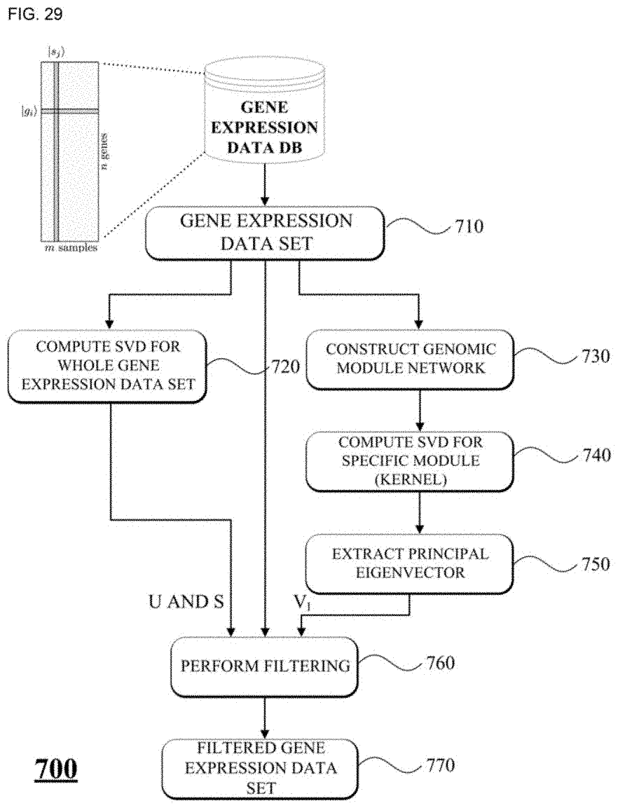

FIG. 29 illustrates an example of process for generating a filtered gene expression dataset 700. The gene expression data DB stores gene expression data to be filtered. The computer apparatus may filter the first gene expression data and second gene expression data, respectively. The computer apparatus removes a specific component of a linear combination from the gene expression data to be filtered. Various techniques may be used to remove the specific component of the linear combination. For convenience of description, it is assumed that singular value decomposition (hereinafter referred to as SVD) is used.

As shown in FIG. 29, the gene expression data DB contains expression data for n genes for each of m samples. The computer apparatus removes the specific component of the linear combination from the gene expression data in the form of a two-dimensional matrix. The SVD decomposes matrix A of m×n as expressed in Equation 16 below. ∪ is a left singular vector matrix, S is a singular value matrix, and V is a right singular vector matrix. The SVD is a technique well known in the art, and thus a detailed description thereof will be omitted.

A=USV^{T} [Equation 16]

[symbol]

A computer apparatus receives a gene expression dataset (710). The computer apparatus performs the SVD on the entire gene expression dataset (720).

The computer apparatus constructs a genomic module network using gene expression data of a normal tissue (730). The computer apparatus selects a specific module for filtering among modules belonging to the constructed genomic module network, and performs the SVD computation on the specific module (740). The computer apparatus performs the SVD computation on gene expression data belonging to the specific module. For convenience of description, it is assumed that the specific module is a kernel module. The computer apparatus extracts a principal eigenvector (a column vector) of V (right singular vector matrix) from the SVD result for the kernel module (750).

The computer apparatus selects ∪ (left singular vector matrix) and S (singular value matrix) for the entire gene expression data. Also, the computer apparatus selects a principal eigenvector V₁ of V (right singular vector matrix) from the SVD result for the kernel module.

The computer apparatus performs filtering using U (left singular vector matrix) and S (singular value matrix) from the SVD results for the entire gene expression data and also the principal eigenvector V₁ of V (right singular vector matrix) from the SVD result for the kernel module (760). Thus, the computer apparatus provides filtered gene expression datasets in a uniform manner (770).

TABLE 5

Calculate filtered expression data G′
1: G₍₁₎ = U₍₁₎S₍₁₎V₍₁₎^{T}: apply SVD on the first module of kernel domain
2: |υ [symbol] ← extract the first column vector of V₍₁₎
3: G = USV^{T}: apply SVD on whole expression data
4: |ƒ [symbol] = US|υ [symbol]
5: normalize |ƒ [symbol]
6: G′ = G − |ƒ [symbol] : subtract |ƒ [symbol] from each column vector of G
7: normalize G′′

[symbol]

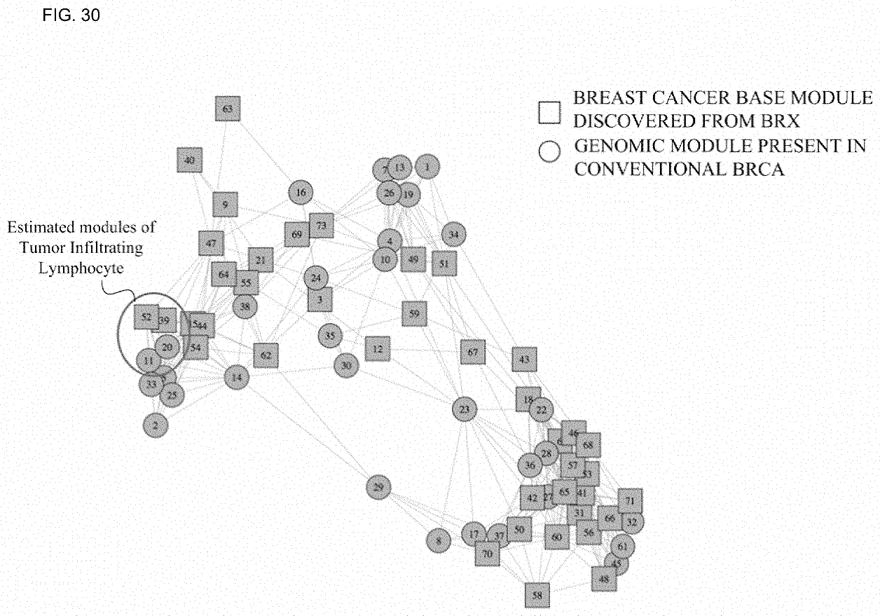

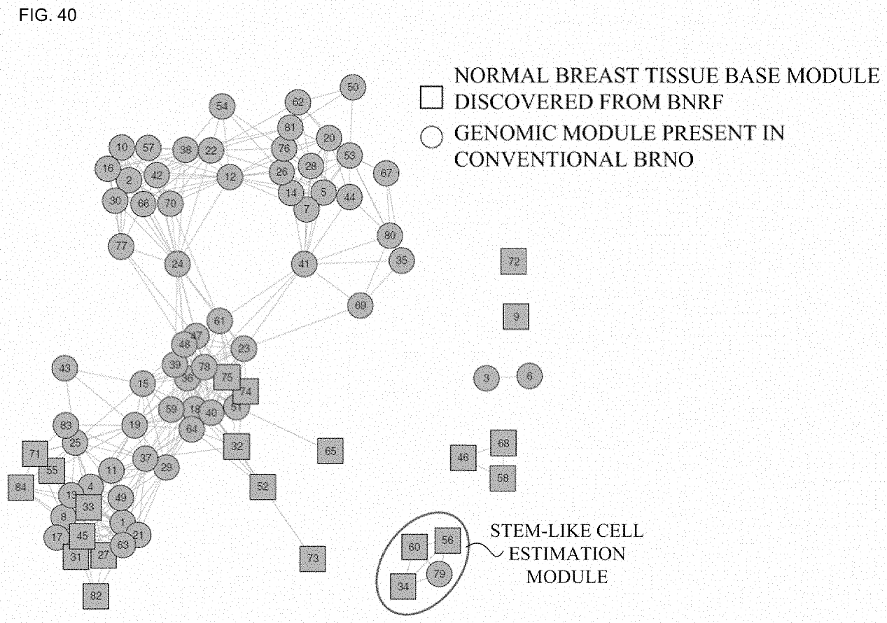

Table 5 above shows pseudocode for the filtering process. Table 5 is an example of filtering process based on the first kernel module of the kernel domain. The computer apparatus generates a filter value vector |f [symbol] by multiplying the first eigenvector V₁ of V (right singular vector matrix) from the SVD result for the kernel module by ∪ (left singular vector matrix) and S (singular value matrix) from the SVD results for the entire gene expression data. In some cases, the computer apparatus normalizes the filter value vector. Finally, the computer apparatus generates G′ obtained by subtracting the filter value vector from each piece of column data of the entire gene expression data G. G′ corresponds to the filtered gene expression data. In some cases, the computer apparatus may normalize G′.

[symbol] Hereinafter, an example of the constructed genomic module network will be described based on the filtered data. Originally, the gene expression data is a value configured through linear combination. When data filtering for removing a specific component is performed, it is possible to determine primary features hidden by the specific component. A new module derived from the filtering result is referred to as a latent genomic module. The genomic module network constructed based on the filtered data is also meaningful for analysis. Two experimental examples will be described. One example is for data obtained by filtering BRCA (breast cancer data). The example for BRCA will be described with reference to FIGS. 30A to 39. The other example is for data obtained by filtering BRNO (normal breast tissue data). The example for BRNO will be described with reference to FIGS. 40 to 42.

[symbol]

FIGS. 30A and 30B illustrate an example of a genomic module network based on a filtered BRCA gene expression dataset. The filtered data of BRCA is referred to as BRX. BRX is a result obtained by filtering BRCA using a principal eigenvector of the kernel module. FIGS. 30A and 30B are an example genomic module network constructed based on BRX. The genomic module network constructed based on BRX is hereinafter referred to as a BRX genomic module network.

In FIGS. 30A and 30B, modules which also present in a genomic module network from BRCA (unfiltered data) are denoted by a circle. In FIGS. 30A and 30B, modules present only in the BRX genomic module network are denoted by a quadrangle. There are latent genomic modules that have not been identified before the filtering. As will be described below, the activity of a module related to a tumor-infiltrating lymphocyte (TIL) among modules of the BRX genomic module network is identified. For example, module #11 (BRX #11), module #20 (BRX #20), module #39 (BRX #39), and module #52 (BRX #52) of the BRX genomic module network contain a large number of various chemokine ligands necessary for lymphocyte migration, interleukin receptors involved in immune responses, interferons, etc. Among the genomic modules, BRX #11 and BRX #20 are genomic modules that are present even in the BRCA genomic module network before the filtering, and BRX #39 and BRX #52 are latent genomic modules that are newly found after the filtering.

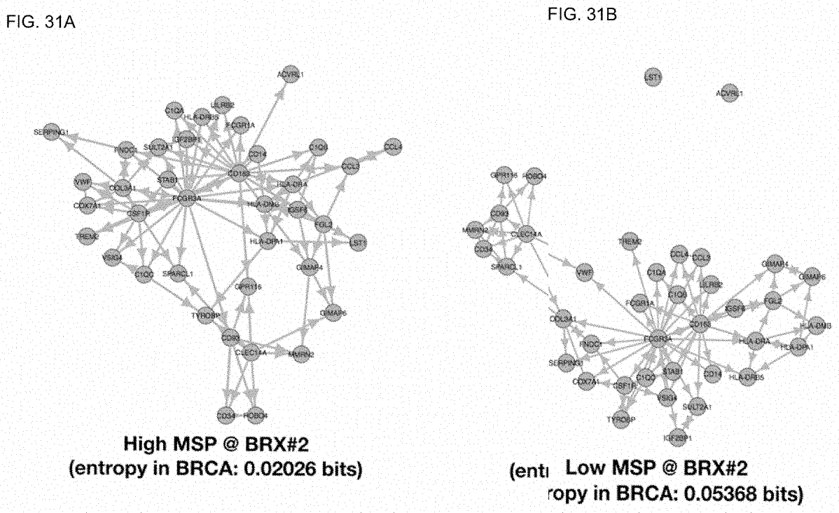

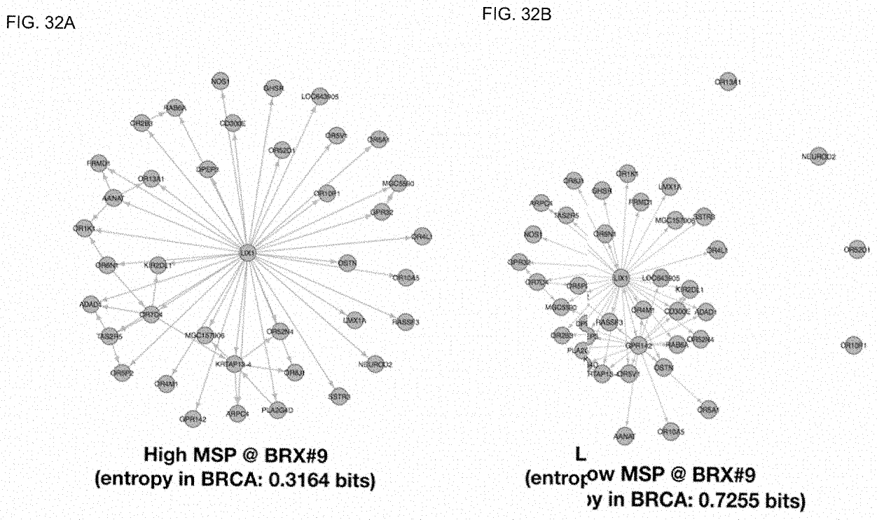

FIGS. 31A and 31B illustrates an example of samples classified based on an MSP at a specific module of the genomic module network in FIGS. 30A and 30B. FIG. 32 illustrates another example of samples classified based on an MSP at a specific module of the genomic module network in FIGS. 30A and 30B. FIGS. 31A, 31B and 32 are examples of the samples which are classified into a high-MSP sample group and a low-MSP sample group based on an MSP maximizing a hazard ratio of Cox proportional hazard model survival analysis. FIGS. 31A and 31B are an example in which samples are classified based on an MSP for module #2 (BRX #2) of the BRX genomic module network. FIGS. 31A and 31B show genetic network of BRX #2 constructed using high-MSP sample group A and low-MSP sample group B for BRX #2. When the entropy of BRX #2 is calculated using the unfiltered data of each sample group, it is confirmed that the entropies of both of the sample groups are very low. This proves that BRX #2 is a genomic module that is also present in the genomic module network before the filtering. FIG. 32 is an example in which samples are classified based on an MSP for module #9 (BRX #9) of the BRX genomic module network. FIG. 32 shows a genetic network of BRX #9 constructed using high-MSP sample group A and low-MSP sample group B for BRX #9. When the entropy of BRX #9 is calculated using the unfiltered BRCA data of each sample group, it is confirmed that the entropy of the low-MSP sample group is two times or more that of the high-MSP sample group. This proves that BRX #9 is a latent genomic module that is not found from the BRCA genomic module before the filtering. FIG. 32 shows that genetic network B of BRX #9 in the low-MSP sample group has better connectivity than genetic network A in the high-MSP sample group.

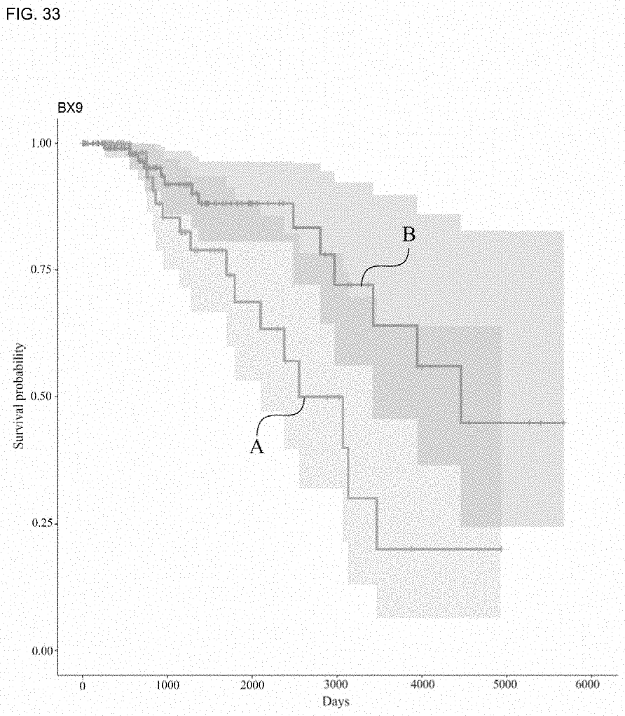

FIGS. 33A and 33B illustrate an example of survival analysis for specific sample groups. FIGS. 33A and 33B are an example in which Kaplan-Meier survival analysis is performed on breast cancer patient groups classified using BRX #9. In FIGS. 33A and 33B, A denotes a survival analysis on a 99% confidence interval of patients in a high-MSP sample group, and B denotes a survival analysis on a 99% confidence interval of patients in a low-MSP sample group. The biological characteristic of BRX #9 derived from the BRX genomic module network have not been fully examined. However, the result of the Cox proportional hazard model survival analysis shows that the survival rate of the patients in the low-MSP sample group was 2.4 times that of the patients in the high-MSP sample group (p-value<0.05). Therefore, BRX #9 is an important genomic module for classification of breast cancer samples with respect to breast cancer progression. FIGS. 33A and 33B suggests that a sample with a low MSP for a BRX latent genomic module is not related with breast cancer, i.e., the sample close to a normal tissue. As a result of classifying patients using MSPs for several different breast cancer latent genomic modules, the survival rate of the patients in the low-MSP sample group was observed to be high.

[symbol]

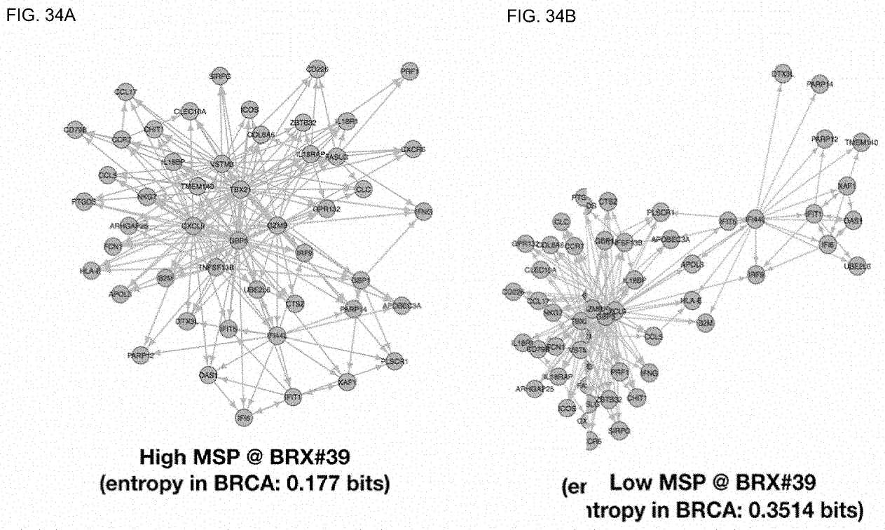

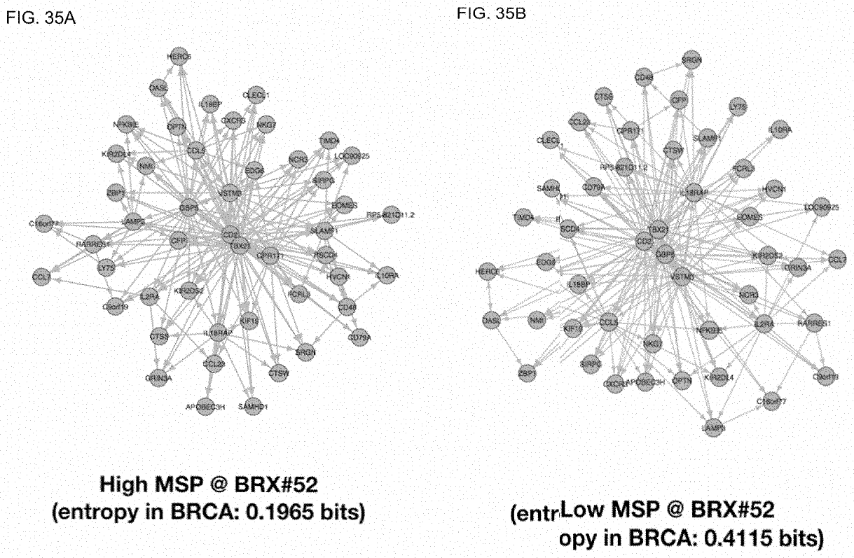

FIGS. 34A and 34B illustrate an example of sample classification based on MSPs at another module of the genomic module network in FIGS. 30A and 30B. FIG. 35 illustrates another example of sample classification based on MSPs at another module of the genomic module network in FIGS. 30A and 30B. FIGS. 34A, 34B and 35 are examples in which samples are classified based on an MSP for a TIL-related BRX latent genomic module of the genomic module network of FIGS. 30A and 30B. FIGS. 34A, 34B and 35 are examples in which when samples are classified based on an MSP for a corresponding BRX genomic module. The samples are classified into a high-MSP sample group and a low-MSP sample group based on an MSP maximizing a hazard ratio of Cox proportional hazard model survival analysis. FIGS. 34A and 34B are an example in which samples are classified on the basis of an MSP for module #39 (BRX #39) of the BRX genomic module network. FIGS. 34A and 34B show genetic network of BRX #39 constructed using high-MSP sample group A and low-MSP sample group B for BRX #39. Like FIG. 32, FIGS. 34A and 34B show that the genetic network of BRX #39 has better connectivity in the low-MSP sample group than in the high-MSP sample group. FIG. 35 shows a genetic network of BRX #52 constructed using high-MSP sample group A and low-MSP sample group B for BRX #52. FIGS. 34A, 34B and 35 show that when the entropy of a corresponding latent genomic module is calculated using the unfiltered BRCA data of each sample group, the entropy of the low-MSP sample group is two times that of the high-MSP sample group.

[symbol]

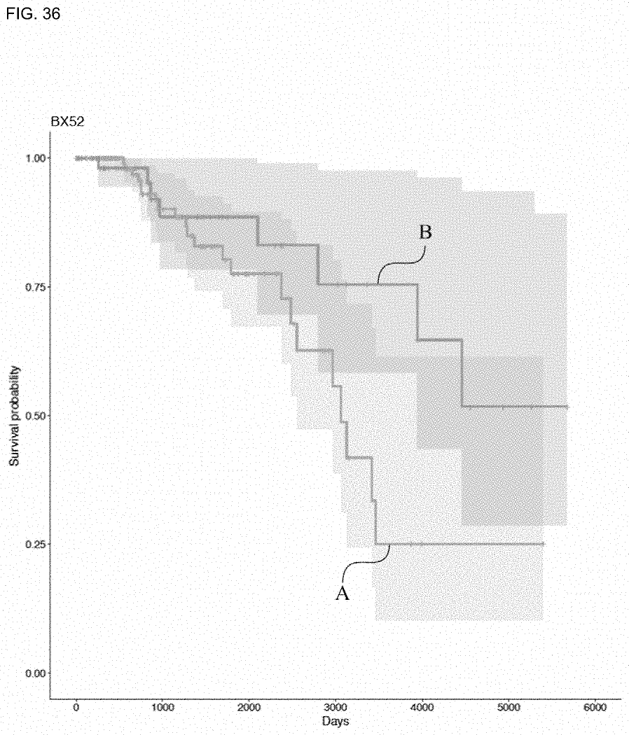

FIGS. 36A and 36B illustrate of survival analysis for sample groups classified in FIG. 35. FIGS. 36A and 36B are an example in which Kaplan-Meier survival analysis is performed on breast cancer patient groups classified using BRX #52. In FIGS. 36A and 36B, A denotes a survival analysis on a 99% confidence interval of patients in a high-MSP sample group, and B denotes a survival analysis on a 99% confidence interval of patients in a low-MSP sample group. The result of the Cox proportional hazard model survival analysis shows that the survival rate of the patients in the low-MSP sample group was about 3 times that of the patients in the high-MSP sample group (p-value<0.05). FIGS. 36A and 36B are an example of a TIL-related BRX latent genomic module for classification of breast cancer samples with respect to breast cancer progression.

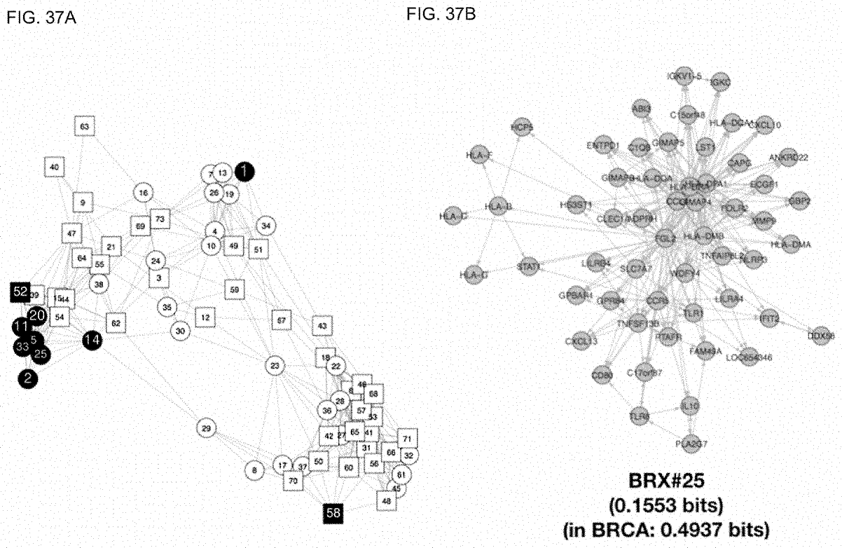

FIGS. 37A and 37B illustrate an example of genomic modules for classifying breast cancer samples of lymphocyte infiltration among the intermodular network in FIGS. 30A and 30B. In FIGS. 37A and 37B, the degree of lymphocyte infiltration of the breast cancer tissue was obtained using data obtained from a tumor tissue clinical information DB. FIG. 37A is an example of a module for classification of breast cancer samples. In FIG. 37A, black symbols denote genomic modules for classifying samples with high lymphocyte infiltration with samples with low lymphocyte infiltration (p-value<0.05). It can be seen that most of the genomic modules are TIL-related modules in the genomic module network of FIGS. 30A and 30B. FIG. 37B shows a genetic network of module #25 (BRX #25) of the BRX genomic module network. FIG. 37B shows that multiple human leukocyte antigen (HLA) genes constituting a major histocompatibility complex (MHC) are included in BRX #25. Especially, with FGL2, which is known as an important gene regulating an immune response, as the center, MHC class II genes (HLA-DMA, HLA-DMB, HLA-DOA, HLA-DPA1, HLA-DQA1, and HLA-DRA) form many edges of the network, and MHC class I genes (HLA-B, HLA-C, HLA-F, and HLA-G) form a small but distinct network. In addition, genes related to chemokine ligands or receptors (CCL4, CCR5, CXCL10, and CXCL13), immunoglobulins (IGKC, IGKV1-5, LILRA4, and LILRB4), immune-related proteins (GIMAP4, GIMAP5, and GIMAP6) are included therein.

[symbol]

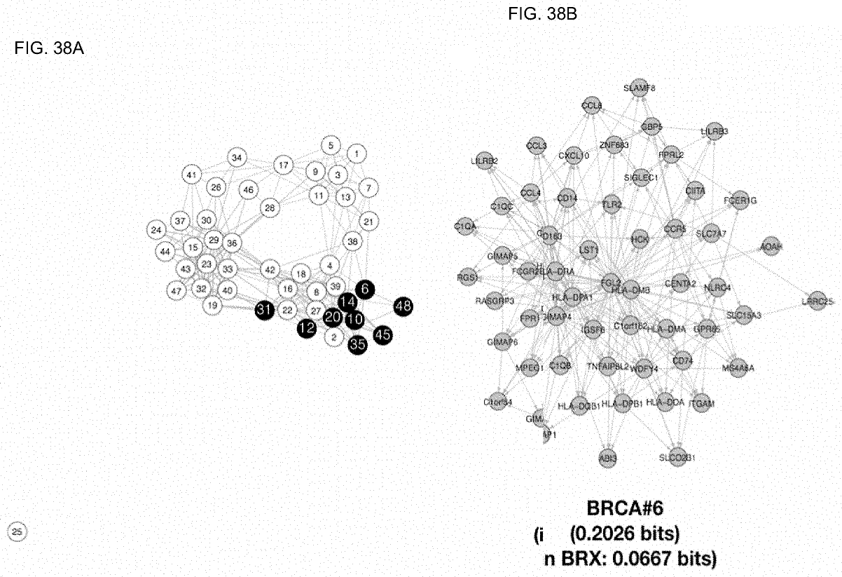

FIGS. 38A and 38B illustrate an example of genomic modules for classifying breast cancer samples of lymphocyte infiltration among the BRCA intermodular network. In FIGS. 38A and 38B, the degree of lymphocyte infiltration of the breast cancer tissue was determined by data obtained from the tumor tissue clinical information DB. FIG. 38A is an example of a module for classification of breast cancer samples. In FIG. 38A, black symbols denote genomic modules for classifying samples with high lymphocyte infiltration and samples with low lymphocyte infiltration (p-value<0.05). FIG. 38B shows a genetic network of module #6 (BRCA #6) of the BRX genomic module network. FIG. 38B shows that BRCA #6 includes multiple genes related to an immune response. In particular, with FGL2, which is known as an important gene regulating an immune response, as the center, HLA-DMA, HLA-DMB, HLA-DOA, HLA-DPA1, HLA-DPB1, HLA-DQB1, HLA-DRA, etc. corresponding to MHC class II form many edges of the genetic network, similarly to BRX #25, but genes corresponding to MHC class I are not included. In addition, genes related to chemokine ligands or receptors (CCL3, CCL4, CCL8, CCR5, and CXCL10), immunoglobulins (FGER1G, FCGR2B, LILRB2, and LILRB3), and immune-related proteins (GIMAP1, GIMAP4, GIMAP5, and GIMAP6) are included therein. FIG. 37B and FIG. 38B show that the configurations of the genes in the genetic networks of BRX #25 and BRCA #6 are partially similar to each other. However, an entropy calculated by mapping BRX #25 to BRCA increases to three times or more while an entropy calculated by mapping BRCA #6 to BRX decreases to a half or less. Accordingly, it is shown that the MSP for the BRX genomic module may be a more accurate reference than the MSP for the BRCA genomic module for breast cancer which are classified with respect to lymphocyte infiltration.

[symbol]

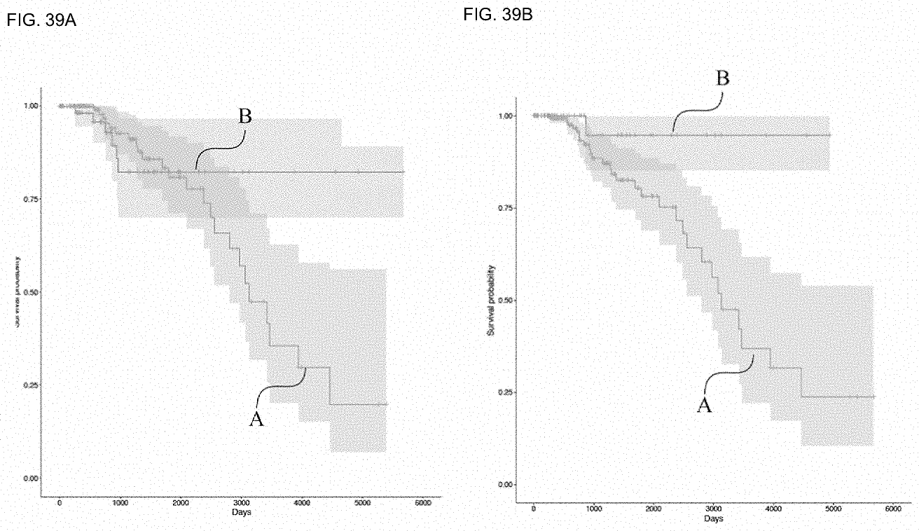

FIG. 39 illustrates an example of survival analysis on sample groups classified using a breast cancer latent genomic module in FIGS. 30A and 30B and a genomic module of different dataset. FIG. 39A is an example in which samples are classified based on an MSP for module #60 (BRX #60), which is one breast cancer latent genomic module, of the BRX genomic module network. In FIG. 39A, A denotes a survival analysis on a 99% confidence interval of patients in a high-MSP sample group, and B denotes a survival analysis on a 99% confidence interval of patients in a low-MSP sample group. The result of the Cox proportional hazard model survival analysis showed that the survival rate of the patients in the low-MSP sample group was about 1.7 times that of the patients in the high-MSP sample group (p-value=0.243). However, this result has low significance and thus there is no difference in survival rate between the two sample groups. FIG. 39A is an example showing that not all BRX latent genomic modules allow for meaningful classification of breast cancer samples in relation to breast cancer progression.

FIG. 39B is an example in which survival rates are analyzed using both of the BRX genomic module network and the genomic module network of a normal breast tissue (BRNO). FIG. 39B is an example in which samples are classified on the basis of an MSP for module #60 (BRX #60) of the BRX genomic module network and an MSP for module #24 (BRNO #24) of the BRNO genomic module network. In FIG. 35B, A denotes a survival analysis on a 99% confidence interval of patients belonging to both of a high-MSP sample group for BRX #60 and a low-MSP sample group for BRNO #24, and B denotes a survival analysis on a 99% confidence interval of patients belonging to both of a low-MSP sample group for BRX #60 and a high-MSP sample group for BRNO #24. The result of the Cox proportional hazard model survival analysis shows that the survival rate of the patients belonging to both of the low-MSP sample group for BRX #60 and the high-MSP sample group for BRNO #24 was about 8.2 times that of the patients belonging to both of the high-MSP sample group for BRX #60 and the low-MSP sample group for BRNO #24 (p-value<0.05). FIG. 39 shows that even a BRX latent genomic module which cannot be used for classification of breast cancer samples with respect to breast cancer progression can be used for classification of the breast cancer samples in a combination with the BRNO genomic module.

[symbol]

FIG. 40 illustrates an example of an intermodular network based on a filtered BRNO gene expression dataset. The data obtained by filtering BRNO is referred to as BNRF. BNRF is a result obtained by filtering BRNO using a principal eigenvector of a kernel module. FIG. 40 is an example genomic module network constructed based on BNRF. The genomic module network constructed based on BNRF is hereinafter referred to as a BNRF genomic module network. In FIG. 40, a module present even in a conventional genomic module network constructed from BRNO (unfiltered data) as well as in the BNRF genomic module network is denoted by a circle. In FIG. 40, a module present only in the BNRF genomic module network is denoted by a quadrangle. Latent genomic modules that have not been identified before the filtering appear. In the BNRF genomic module network, a stem cell-like cell estimation module that had been found in the genomic module network of the breast cancer (BRCA) but had not been found in the genomic module network of the normal breast tissue (BRNO) is identified as a latent genomic module.

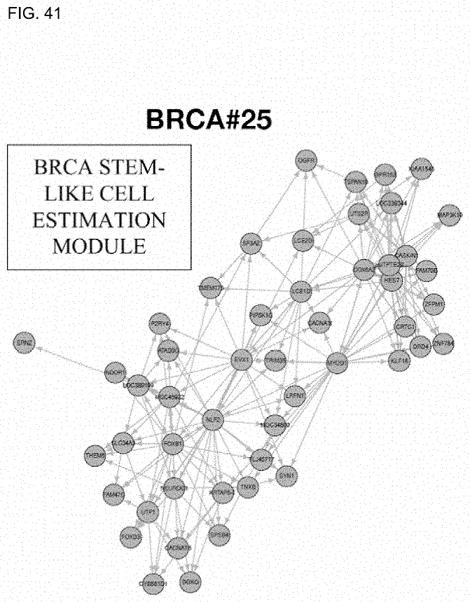

FIGS. 41A and 41B illustrate an example of a genomic module of a stem cell-like cell in the BRCA genomic module network. FIGS. 41A and 41B are an example of module #25 (BRCA #25) of the genomic module network constructed from BRCA. BRCA #25 is a stem cell-like cell estimation module extracted from data on breast cancer patients. FIGS. 41A and 41B show that the genetic network of BRCA #25 is composed of several genes (CACNA1B, EVX1, FOXD3, HES7, KLF16, LCE1D, NEUROG1, NLF2, P2RY4, UTF1, etc.) playing important roles during development of embryos or differentiation of neural progenitor cells. Genomic modules similar to BRCA #25 are found in most tumor tissue genomic module networks, including colon adenocarcinoma (COAD), rectum adenocarcinoma (READ), lung adenocarcinoma (LUAD), lung squamous cell carcinoma (LUSC), ovarian serous cystadenocarcinoma (OV), and the like, and also are found even in the CONO (normal colon tissue) genomic module network. On the contrary, the similar genomic modules are not found in the BRNO (normal breast tissue) genomic module network.

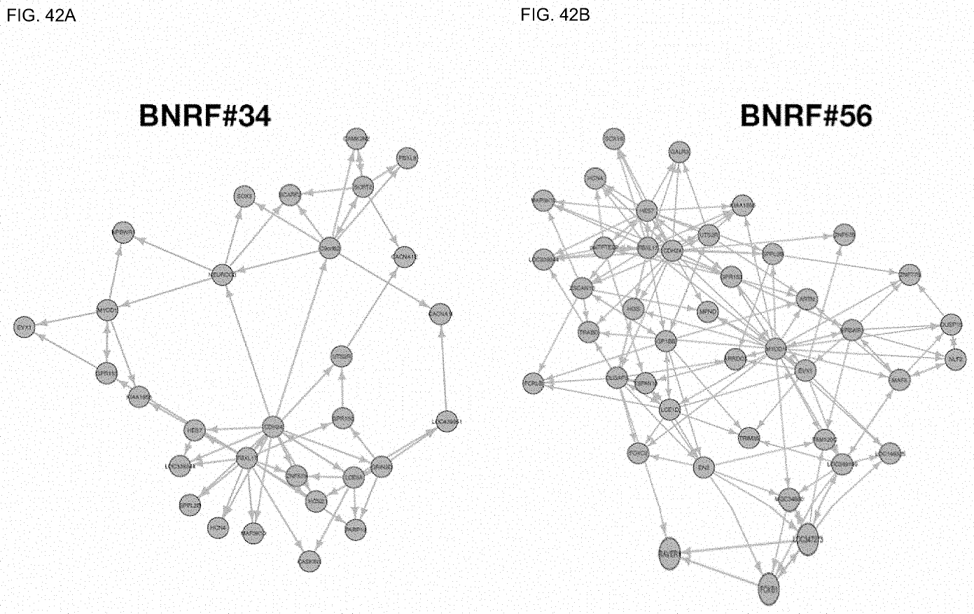

FIG. 42 illustrates an example of estimated modules of a stem cell-like cell in BNRF genomic module network. FIG. 42 is an example showing a module similar to BRCA #25 in the BNRF genomic module network. FIG. 42A is an example of module #34 (BNRF #34) of the BNRF genomic module network. FIG. 42B is an example of module #56 (BNRF #56) of the BNRF genomic module network. FIG. 42A shows that the genetic network of BNRF #34 includes multiple genes (CACNA1I, CASKIN1, EVX1, GPR153, HES7, MAPK3K10, MYOD1, and UTS2R) included in BRCA #25. FIG. 42B shows that the genetic network of BNRF #56 includes multiple genes (EVX1, FOXB1, GPR153, HES7, LCE1D, MAPK3K10, MYOD1, NLF2, TRIM35, TSPAN10, and UTS2R) included in BRCA #25.

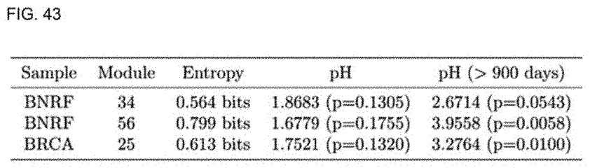

FIG. 43 illustrates an example of samples classification based on MSPs at the genomic module network in FIGS. 41A, 41B and FIG. 42. FIG. 43 is an example in which when samples are classified on the basis of an MSP for a specific BNRF genomic module, the samples are classified into a high-MSP sample group and a low-MSP sample group on the basis of an MSP maximizing a hazard ratio of Cox proportional hazard model survival analysis. FIG. 43 is a result of Kaplan-Meier survival analysis of breast cancer patient groups classified based on MSPs for BNRF #34 and BNRF #56, which are stem cell-like cell estimation modules of BNRF, and the survival analysis result of the breast cancer patient groups classified on the basis of an MSP for BRCA #25. The result of the Cox proportional hazard model survival analysis using the entire survival period shows that the survival rates of patients in high-MSP sample groups for BNRF #34, BNRF #56, and BRX #25 are about 1.9 times (p-value=0.13), about 1.7 times (p-value=0.18), and about 1.8 times (p-value=0.13) those of patients in low-MSP sample groups, respectively. However, this result has low significance and thus there is no difference in survival rate between the two sample groups. Meanwhile, the result of the Cox proportional hazard model survival analysis using a survival period after 900 days shows that the survival rates of the patients in the high-MSP sample groups for BNRF #34, BNRF #56, and BRX #25 were about 2.7 times (p-value<0.1), about 4 times (p-value<0.01), and about 3.3 times (p-value<0.01) those of the patients in the low-MSP sample groups, respectively.

FIGS. 41A to 43 show that if the gene expression data of the normal breast tissue is filtered using a principal eigenvector of a kernel module of the normal breast tissue, modules corresponding to the stem cell-like cell estimation module commonly observed in the normal colon tissue and various tumor tissues including breast cancer can be identified in the normal breast tissue. This shows that a genomic module hidden by expression of the kernel module genes may be revealed through filtering. This is consistent with the fact that a latent genomic module capable of accurately classifying breast cancer patients is found by filtering the gene expression data of the breast cancer tissue using the principal eigenvector of the breast cancer kernel module in FIGS. 31A to 39. Accordingly, it is confirmed that the construction of a genomic module network with data filtered using a principal eigenvector of a specific genomic module is a method of discovering a latent genomic module hidden by activation of corresponding modules of the normal breast tissue and the breast cancer tissue.

[symbol]

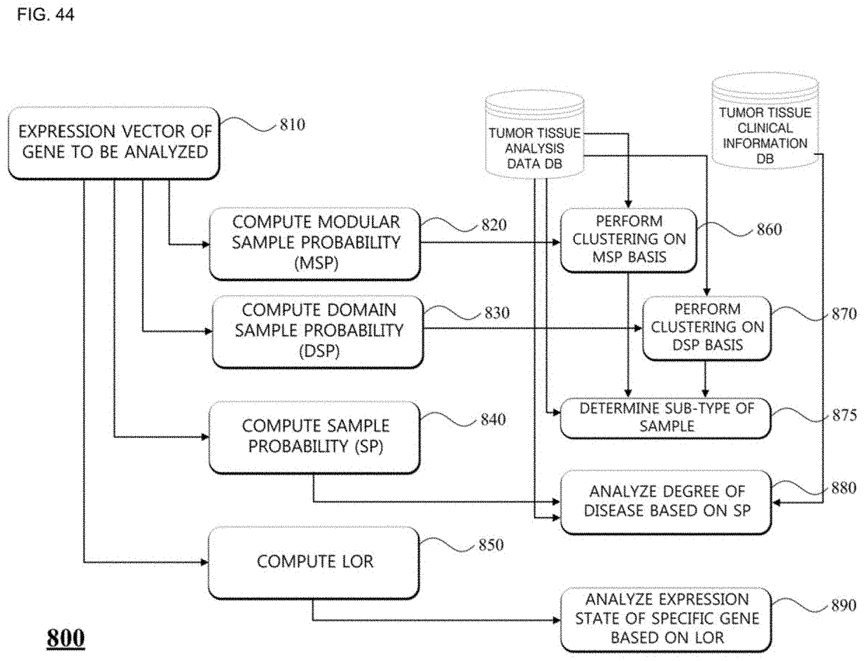

FIGS. 44A-44C illustrate an example of a sample data analysis process 800 based on a genomic module network. FIGS. 44A-44C are an example in which samples are analyzed using various indicators derived in FIGS. 16 to 28.

The process of FIGS. 44A-44C are performed by a computer apparatus. It is assumed that necessary data is provided in advance through the processes of FIGS. 16 to 28. The computer apparatus provides an SP, MSP, DSP, and LOR, which have been described above, using the gene expression data of a tumor tissue and a normal tissue in advance. The computer apparatus stores an SP, MSP, DPS, and LOR provided in advance for a specific tissue by a tumor tissue analysis data DB. Also, a tumor tissue clinical information DB stores clinical information on patients. For example, the tumor tissue clinical information DB may include information regarding whether patients have died and their dates of death, which is used to determine the survival rate in FIGS. 18A and 18B, information regarding whether an ER, a PR, and an HER2 are expressed, which is used to interpret an MSP-based clustering result in FIG. 20, etc.

The computer apparatus acquires a gene expression vector of a sample to be analyzed (810). The computer apparatus configures the gene expression vector to be composed of the same genes as those used to calculate a density matrix. The computer apparatus computes an MSP 820, a DSP 830, an SP 840, and an LOR 850 on an input sample through the above-described process.

The computer apparatus may perform, in advance, clustering on the corresponding sample on the basis of an MSP of a tumor tissue reference sample. That is, the computer apparatus classifies the gene expression data on an MSP basis in a uniform manner. The clustering result based on the MSP for the tumor tissue reference sample is referred to as a first reference cluster. The computer apparatus may determine an MSP of the sample to be analyzed (820) and may classify the sample to be analyzed using the first reference cluster constructed in advance on the basis of the MSP (860). Alternatively, in some cases, the computer apparatus may integrate the MSP of the sample to be analyzed and the MSP of the tumor tissue reference sample constructed in advance, and then perform the clustering (860). The MSP clustering is for determining to which aspect of tumor expression a sample (patient) belongs (875). The MSP clustering is a process of determining a specific sub-type for the sample (patient).

The computer apparatus may perform, in advance, clustering on the corresponding sample on the basis of a DSP of the tumor tissue reference sample. That is, the computer apparatus classifies the gene expression data on a DSP basis in a uniform manner. The clustering result based on the DSP for the tumor tissue reference sample is referred to as a second reference cluster. The computer apparatus may determine the MSP of the sample to be analyzed (830) and may classify the sample to be analyzed using the second reference cluster constructed in advance on the basis of the MSP (870). Alternatively, in some cases, the computer apparatus may integrate the DSP of the sample to be analyzed and the DSP of the tumor tissue reference sample constructed in advance, and then perform the clustering (860). The DSP clustering is for determining to which aspect of tumor expression a sample (patient) belongs (875). The DSP clustering is a process of determining a specific sub-type for the sample (patient).

The computer apparatus may perform clustering (classification) on the sample to be analyzed according to the MSP and DSP and may utilize clinical information stored in the tumor tissue clinical information DB to interpret a result of the classification.

In FIGS. 44A-44C, the computer apparatus may compare an SP of the sample to be analyzed and an SP of the tumor tissue reference data to predict the degree of disease in the corresponding patient (880). The computer apparatus may predict the degree of disease in the patient on an SP basis by utilizing the clinical information stored in the tumor tissue clinical information DB.

Furthermore, the computer apparatus may compare an LOR of each gene calculated from the sample to be analyzed and an LOR of each gene calculated from the tumor tissue reference data and then analyze a gene specifically affecting the variation of the genomic system of the sample to be analyzed (890).

[symbol]

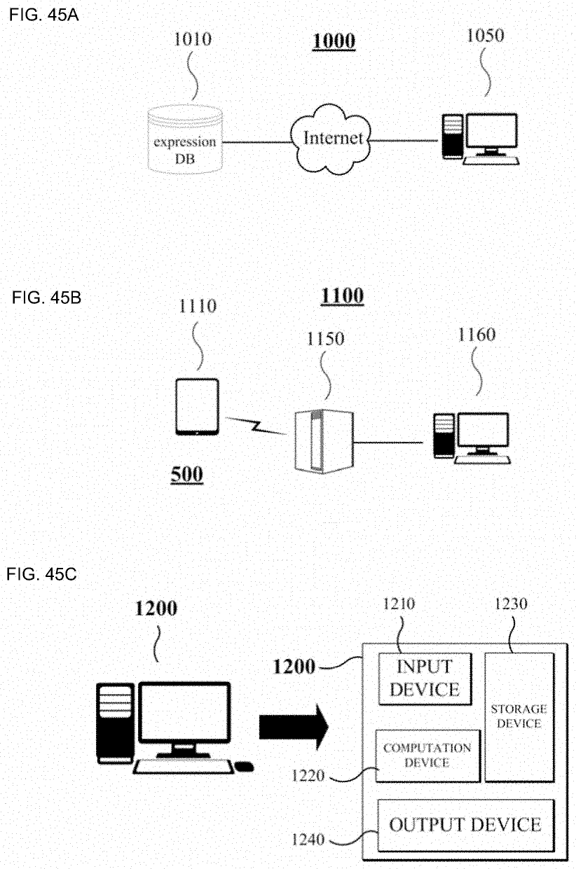

FIGS. 45A-45C illustrate an example of a sample data analysis apparatus based on genomic module network. FIGS. 45A-45C show a computer apparatus for constructing a genomic module network and analyzing gene expression data.

FIG. 45A is a system 1000 for constructing a genomic module network using gene expression data provided in advance and analyzing samples. The system 1000 includes a gene expression DB 1010 and a computer apparatus 1050.

The gene expression DB 1010 stores data related to gene expression of a specific living thing. As described above, the gene expression data is generated using a technique such as cDNA microarray. The gene expression DB 1010 may be expression data associated with a specific disease (e.g., a malignant tumor).

As described above, the computer apparatus 1050 constructs the genomic module network using the expression data stored in the gene expression DB 1010. Also, the computer apparatus 1050 analyzes sample data on the basis of the genomic module network.

A researcher can analyze a genome or gene on the basis of the genomic module network constructed by the computer apparatus 1050. Furthermore, the researcher can construct a genomic module network for a specific patient, compare the constructed genomic module network with a normal genomic module network to be compared, and diagnose the patient.

FIG. 45B is another example of a system 1100 for constructing a genomic module network and analyzing sample data. The system 1100 includes a researcher terminal 1110, a central server 1150, and a user terminal 1160.

A researcher conducts an experiment on gene expression for a patient and inputs a result of the gene expression to the researcher terminal 1110. For example, the researcher conducts a microarray experiment on mRNA of a patient and stores data associated with expression in the researcher terminal 1110. The data includes text, images, etc. When the data is an image, software for analyzing the image may be used. The researcher terminal 1110 transfers input data to the central server 1150. The central server 1150 stores and manages gene expression data for a specific patient.

The central server 1150 may construct a specific genomic module network using the gene expression data. Furthermore, the central server 1150 may provide information regarding diagnosis or treatment for a patient on the basis of the constructed genomic module network. In this case, the central server 1150 corresponds to a computer apparatus for constructing the genomic module network and analyzing sample data. The user terminal 1160 may access the central server 1150 as a client apparatus to check the genomic module network or check information regarding diagnosis or the like.

Furthermore, the central server 1150 may store and manage the gene expression data for the specific patient. In this case, the user terminal 1160 analyzes expression data stored in the central server 1150 and constructs the genomic module network for the specific living thing. Furthermore, the user terminal 1160 may provide information regarding diagnosis or treatment for a patient on the basis of the constructed genomic module network.

FIG. 45C is an example of an apparatus 1200 for constructing a genomic module network and analyzing sample data. FIG. 45C is an example in which a computer apparatus drives a specific program to construct or analyze a genomic module network. As an example, FIG. 40C shows an apparatus such as a personal computer (PC), but the apparatus 1200 of FIG. 45C may be a server apparatus such as the central server 1150. The genomic module network construction apparatus 1200 includes an input device 1210, a computation device 1220, a storage device 1230, and an output device 1240.

The input device 1210 receives gene expression data. The input device 1210 may be a physical interface device such as a keyboard, a mouse, and a touch pad. Alternatively, the input device 1210 may be a device for receiving stored gene expression data from an external storage medium (a Universal Serial Bus (USB)). Alternatively, the input device 1210 may be a communication module for receiving the gene expression data from an external network.

The storage device 1230 stores a program for constructing the genomic module network. Also, the storage device 1230 may store a program for performing specific analysis using the genomic module network. The program stored in the storage device 1230 may store source code for constructing the genomic module network for genes or source code for analyzing sample data according the above description.

The computation device 1220 performs computation for constructing the genomic module network using the program stored in the storage device 1230 and the input gene expression data. Furthermore, the computation device 1220 may perform a process of analyzing the constructed genomic module network using an analysis program stored in the storage device 1230. The computation device 1220 refers to a processor device for processing specific computation through a program, such as a central processing unit (CPU) and an application processor (AP).

The output device 1240 is a device for outputting the constructed genomic module network and the analysis result. The output device 1240 may be a display device for outputting images, a printer for outputting text, or the like. Furthermore, the output device 1240 may be a communication module for transferring the generated genomic module network or the analysis data to another apparatus.

Also, the genomic module network construction method, the genomic module network-based sample data analysis method, and the sample data analysis method based on the genomic module network configured using the filtered data, which have been described above, may be implemented as a program (or an application) including an algorithm executable on a computer. The program may be stored and provided in a non-transitory computer-readable medium.

The non-transitory computer-readable medium refers not to a medium that temporarily stores data such as a register, a cache, and a memory but to a medium that semi-permanently stores data and that is readable by a device. Specifically, the above-described various applications or programs may be provided while being stored in a non-transitory computer-readable medium such as a compact disc (CD), a digital versatile disc (DVD), a hard disk, a Blu-ray disc, a Universal Serial Bus (USB), a memory card, a read-only memory (ROM), etc.

1-24. (canceled)

25. A method of analyzing sample data based on a genomic module network by an analysis apparatus, the method comprising:
generating, by the analysis apparatus, the genomic module network comprising a plurality of genomic modules based on an entropy for a plurality of gene sets using first gene expression data for reference tissues, wherein the reference tissues are either normal or tumorous tissues;
acquiring, by the analysis apparatus, second gene expression data for a sample tissue; and
determining, by the analysis apparatus, a first degree of transformation of the sample tissue relative to the reference tissues by first genes of the reference tissues and second genes of the sample tissue, wherein the first genes and the second genes belong to at least one module of the plurality genomic modules respectively,
wherein the entropy indicates an average information content for interrelationships between two or more genes based on probabilities for transcriptional states of the two or more genes.

26. The method of claim 25, wherein the generating the genomic module network comprises:
dividing randomly a plurality of genes of the reference tissues into a plurality of sets;
removing at least one gene to adjust the entropy of a set to be lower than a threshold value for the plurality of sets respectively; and
adding at least one gene which does not belong to the set on condition that the entropy of the set is less than or equal to the threshold value and a fluctuation of a principal eigenvector of the set is less than or equal to a reference value for the plurality of sets respectively.

27. The method of claim 25, wherein the determining the first degree of transformation comprises:
generating a density matrix in a gene space using the first gene expression data, constructing an expression vector using the second gene expression data, and determining the first degree of transformation using the expression vector and the density matrix.

28. The method of claim 25, wherein the first degree of transformation is computed by Pᵢ below:
$${P}_{i} = P ( {s}_{i} \vert {G}_{M} ) = {σ}_{iM}^{⊤} {ρ}_{M}^{( s )} {σ}_{iM} . \\ {ρ}_{M}^{( s )} = \frac{{G}_{M} {G}_{M}^{⊤}}{t ( {G}_{M} {G}_{M}^{⊤} )} . \\ {σ}_{iM} = \frac{{s}_{Im}}{‖ {s}_{iM} ‖}$$
wherein Pᵢ denotes a degree of transformation of the sample tissue i based on the reference tissues, G_{M} denotes an expression matrix of a set of all genes included in at least one genomic module of the reference tissues, and s_{iM} is an expression vector configured by identifying genes included in the gene set from the gene expression data of the sample tissue, sᵢ.

29. The method of claim 25, further comprising:
determining a second degree of transformation of the target sample tissue relative to the reference tissues by third genes of the reference tissues and fourth genes of the sample tissue, wherein the third genes are genes which excludes a specific gene from the first genes, and the fourth genes are genes which excludes the specific gene from the second genes; and
calculating a value obtained by comparing the first degree of transformation and the second degree of transformation.

30. The method of claim 29, wherein the value is calculated as a log odds ratio (LOR) on the basis of the first degree of transformation and the second degree of transformation.

31. The method of claim 29, the first degree of transformation and the second degree of transformation are for one module of the plurality genomic modules, one domain of the plurality genomic modules or all genes included in the plurality genomic modules, wherein the domain comprises two or more modules of the plurality genomic modules.

32. A method of analyzing sample data based on a genomic module network by an analysis apparatus, the method comprising:
inputting, by the analysis apparatus, a sample gene expression data for a sample tissue;
identifying, by the analysis apparatus, genes of a plurality of genomic modules in the genomic module network from the sample gene expression data; and
analyzing, by the analysis apparatus, the sample tissue by determining a first degree of transformation of the sample tissue relative to reference tissues,
wherein the genomic module network comprising the plurality of genomic modules based on an entropy for a plurality of gene sets using a reference gene expression data for the reference tissues, wherein the reference tissues are either normal or tumorous tissues, and
wherein the first degree of transformation is determined by first genes of the reference tissues and second genes of the sample tissue, wherein the first genes and the second genes belong to at least one module of the plurality genomic modules respectively.

33. The method of claim 32, further comprising generating the genomic module network, wherein the generating the genomic module network comprises:
dividing randomly a plurality of genes of the reference tissues into a plurality of sets;
removing at least one gene to adjust the entropy of a set to be lower than a threshold value for the plurality of sets respectively; and
adding at least one gene which does not belong to the set on condition that the entropy of the set is less than or equal to the threshold value and a fluctuation of a principal eigenvector of the set is less than or equal to a reference value for the plurality of sets respectively.

34. The method of claim 32, wherein the analysis apparatus generates a density matrix in a gene space using the reference gene expression data, constructs an expression vector using the sample gene expression data, and determines the first degree of transformation using the expression vector and the density matrix.

35. The method of claim 32, wherein the first degree of transformation is computed by Pᵢ below:
$${P}_{i} = P ( {s}_{i} \vert {G}_{M} ) = {σ}_{iM}^{⊤} {ρ}_{M}^{( s )} {σ}_{iM} . \\ {ρ}_{M}^{( s )} = \frac{{G}_{M} {G}_{M}^{⊤}}{t ( {G}_{M} {G}_{M}^{⊤} )} . \\ {σ}_{iM} = \frac{{s}_{Im}}{‖ {s}_{iM} ‖}$$
wherein Pᵢ denotes a degree of transformation of the sample tissue i based on the reference tissues, G_{M} denotes an expression matrix of a set of all genes included in at least one genomic module of the reference tissues, and s_{iM} is an expression vector configured by identifying genes included in the gene set from the gene expression data of the sample tissue, sᵢ.

36. The method of claim 32, further comprising:
determining a second degree of transformation of the target sample tissue relative to the reference tissues by third genes of the reference tissues and fourth genes of the sample tissue, wherein the third genes are genes which excludes a specific gene from the first genes, and the fourth genes are genes which excludes the specific gene from the second genes; and
calculating a value obtained by comparing the first degree of transformation and the second degree of transformation.

37. The method of claim 36, wherein the value is calculated as a log odds ratio (LOR) on the basis of the first degree of transformation and the second degree of transformation.

38. The method of claim 36, the first degree of transformation and the second degree of transformation are for one module of the plurality genomic modules, one domain of the plurality genomic modules or all genes included in the plurality genomic modules, wherein the domain comprises two or more modules of the plurality genomic modules.

39. An analysis apparatus for analyzing sample data using a genomic module network by an analysis apparatus, the analysis apparatus comprising:
an input device configured to input a sample gene expression data for a sample tissue using the genomic module network;
a storage device configured to store a program for analyzing the sample gene expression data;
a processor executing the program configured to
identify genes of a plurality of genomic modules in the genomic module network from the sample gene expression data; and
analyze the sample tissue by determining a first degree of transformation of the sample tissue relative to reference tissues,
wherein the genomic module network comprising the plurality of genomic modules based on an entropy for a plurality of gene sets using a reference gene expression data for the reference tissues, wherein the reference tissues are either normal or tumorous tissues, and
wherein the first degree of transformation is determined by first genes of the reference tissues and second genes of the sample tissue, wherein the first genes and the second genes belong to at least one module of the plurality genomic modules respectively.

40. The analysis apparatus of claim 39,
wherein the storage device further configured to store a program for generating the genomic module network, and
wherein the processor further configured to generate the genomic module network by
dividing randomly a plurality of genes of the reference tissues into a plurality of sets;
removing at least one gene to adjust an entropy of a set to be lower than a threshold value for the plurality of sets respectively; and
adding at least one gene which does not belong to the set on condition that the entropy of the set is less than or equal to the threshold value and a fluctuation of a principal eigenvector of the set is less than or equal to a reference value for the plurality of sets respectively.

41. The analysis apparatus of claim 39, wherein the processor further configured to generate the first degree of transformation by
generating a density matrix in a gene space using the reference gene expression data,
constructing an expression vector using the sample gene expression data, and
determining the first degree of transformation using the expression vector and the density matrix.

42. The analysis apparatus of claim 39, wherein the first degree of transformation is computed by Pᵢ below:
$${P}_{i} = P ( {s}_{i} \vert {G}_{M} ) = {σ}_{iM}^{⊤} {ρ}_{M}^{( s )} {σ}_{iM} . \\ {ρ}_{M}^{( s )} = \frac{{G}_{M} {G}_{M}^{⊤}}{t ( {G}_{M} {G}_{M}^{⊤} )} . \\ {σ}_{iM} = \frac{{s}_{Im}}{‖ {s}_{iM} ‖}$$
wherein Pᵢ denotes a degree of transformation of the sample tissue i based on the reference tissues, G_{M} denotes an expression matrix of a set of all genes included in the at least one genomic module of the reference tissues, and s_{iM} is an expression vector configured by identifying genes included in the gene sets from the gene expression data of the sample tissue, sᵢ.

43. The analysis apparatus of claim 39, wherein the processor further configured to
determine a second degree of transformation of the target sample tissue relative to the reference tissues by third genes of the reference tissues and fourth genes of the sample tissue, wherein the third genes are genes which excludes a specific gene from the first genes, and the fourth genes are genes which excludes the specific gene from the second genes; and
calculate a value obtained by comparing the first degree of transformation and the second degree of transformation.

44. The analysis apparatus of claim 43, wherein the value is calculated as a log odds ratio (LOR) on the basis of the first degree of transformation and the second degree of transformation.

45. The method of claim 43, the first degree of transformation and the second degree of transformation are for one module of the plurality genomic modules, one domain of the plurality genomic modules or all genes included in the plurality genomic modules, wherein the domain comprises two or more modules of the plurality genomic modules.
